(19) United States
(12) Patent Application Publication
(10) Pub. No.: US 2025/0281451 A1
(43) Pub. Date: Sep. 11, 2025

(54) COMPOSITIONS AND METHODS FOR MODULATING SLEEP AND USES THEREOF

(71) Applicant: Brightseed, Inc., South San Francisco, CA (US)

(72) Inventors: Nicholas Woodward, San Francisco, CA (US); Chuan Wang, Belmont, CA (US); Pranatchareeya Chankhamjon, South San Francisco, CA (US); Meng Xu, San Mateo, CA (US); Lee Heil Chae, San Francisco, CA (US); James Flatt, Colorado Springs, CO (US); Sadie Barr, Bozeman, MT (US)

(21) Appl. No.: 19/216,032

(22) Filed: May 22, 2025

Related U.S. Application Data
(63) Continuation of application No. PCT/US2023/081013, filed on Nov. 22, 2023.
(60) Provisional application No. 63/384,854, filed on Nov. 23, 2022.
(60) Provisional application No. 63/494,995, filed on Apr. 7, 2023.
(60) Provisional application No. 63/508,807, filed on Jun. 16, 2023.

(51) Int. Cl. A61K 31/365; A61K 9/00; A61K 9/48; A61K 36/28

(52) U.S. Cl. CPC A61K 31/365; A61K 9/0053; A61K 9/4858; A61K 9/4866; A61K 36/28; A61K 2236/15; A61K 2236/39

(57) ABSTRACT

Disclosed herein are compositions and methods for improving a sleep health benefit by providing a composition and a carrier. Some embodiments provided include, for example, administering a therapeutically effective compound of a plant extract. In some embodiments, the plant extract is Eupatorium perfoliatum. In some embodiments, the plant extract includes costunolide.

### CROSS-REFERENCE TO RELATED APPLICATIONS

This application is a continuation of PCT Application No. PCT/US2023/081013, filed Nov. 22, 2023, and claims the benefit of U.S. Provisional Application No. 63/508,807, filed Jun. 16, 2023, U.S. Provisional Application No. 63/494,995, filed Apr. 7, 2023, and U.S. Provisional Application No. 63/384,854, filed Nov. 23, 2022, all of which are incorporated by reference in their entirety.

### FIELD OF THE DISCLOSURE

The present disclosure relates to compositions and their methods of preparation. More particularly, the present disclosure relates to compositions and methods for modulating sleep by administering a therapeutically effective amount of the oral composition and a carrier.

### BACKGROUND

Good sleep is critical for good mental and physical health and quality of life. Insufficient sleep is associated with a range of negative health and social outcomes, including adverse performance at school and in the workplace. Reduced sleep duration has been linked to 7 of the 15 leading causes of death in the U.S., including cardiovascular disease, malignant neoplasm, cerebrovascular disease, accidents, diabetes, septicemia, and hypertension.

Given the prevalence of chronic diseases related to insufficient sleep now afflicting the world's population, an invention that ameliorates or reduces the adverse effects of insufficient sleep would be of immediate and immense benefit to global human health. Good sleep hygiene can encourage optimal sleep duration and quality, such as consistent bed and wake times, reducing caffeine intake, and managing screen time before bed.

Therapeutic interventions are the most common treatment for the treatment of sleep disorders. Currently, there are three FDA-approved molecules—suvorexant, lemborexant, and daridorexant—for the treatment of insomnia through orexin receptor antagonism. Several molecules are being investigated for insomnia. Most therapeutic solutions, however, are not suitable for daily consumption as a nutritional intervention.

Various herbal remedies and dietary supplements are promoted as aids for sleep. These include melatonin, valerian root, Gennan chamomile, kava, L-tryptophan, littleleaf linden (Tilia), vervain (verbena), lemon balm (melissa), and skullcap (Scutellaria). However, these products are not consistently regulated, leading to variations in ingredient purity and effectiveness. Scientific evidence supporting the effectiveness of these herbal solutions is limited. Most are generally considered safe, but there's a lack of information about how they might interact with prescription drugs. Among these, melatonin is the most popular for treating insomnia. It is typically taken in doses of 3 to 5 mg a few hours before bedtime for the best effect, especially when used occasionally. However, it is not recommended for long-term management of chronic insomnia. The long-term safety of melatonin is not known.

Clearly, there remains an unmet need to develop a nutritional intervention for the improvement of sleep quality and duration.

### SUMMARY OF THE DISCLOSURE

Aspects of the disclosure are related to an oral composition for improving, restoring, modulating, or maintaining sleep. In some embodiments, the oral composition includes a therapeutically effective amount of a plant extract, and one or more carriers or excipients, wherein, the plant extract is capable of improving, restoring, modulating, or maintaining sleep. In some embodiments, the plant extract is selected from the group consisting of Eupatorium perfoliatum, Zingiber officinale, Picea sitchensis, Viburnum davidii, Amica montana, Helianthus annuus, Lactuca sativa, Barnadesia spinosa, Dolomiaea souliei, Saussurea costus, Artemisia annua, Inula helenium, Laurus nobilis, Momodica cochinchinensis, Pogostemon cablin, Tanacetum parthenium, Cichorium intybus, Solidago Canadensis, Horeum vulgare, Tricum aestivum, Silene conica, Alloteropsis semialata, Paris polyphylla, Camella sinensis, Glebionis cornaria, Hordeum vulgare, Setaria italic, Zea mays, Arabidopsis thaliana, Brachypodium distachyon, Grindelia integrifolia, Lobelia chinesis, Arachis hypogaea, Nepenthes khasiana, Selaginella mollendorffii, Taxus×media, Acorus gramieus, Andrographis paniculata, Artemisia maritima, Bletilla striata, Fallopia mulyfora, Gossypium hirsutum, Hordeum spontaneum, Hypericum perforatum, Musa acuminate, Oryza rufipogon, Oryza sativa, Quercus suber, Solanum tuberosum, Valeriana officinallis, Vitis vinifera, Polygonatum odoratum, Glycine max, Panicum virgatum cv. Trailblazer, or Populus trichorarpa. In some embodiments, the plant extract comprises a compound selected from the group consisting of costunolide, 5a,9-dimethyl-3-methylidene-3ah,4h,5h,9bh-naphtho[1,2-b]furan-2,8-dione, Baynol A, (3r,3ar,4r,11as)-3,6,10-trimethyl-2-oxo-3h,3ah,4h,7h,8h,11h,11ah-cyclodeca[b]furan-4-yl acetate, (3r,3as,4r,11as)-4-hydroxy-3,6,10-trimethyl-3h,3ah,4h,7h,8h,11h,11ah-cyclodeca[b]furan-2-one, 1R,2S,5S,6S,7S)-5-isopropyl-2,8-dimethyltricyclo[4.4.0.0^{2,7}]dec-8-en-4-one, 2-((1R,3S,4R)-4-methyl-3-(prop-1en-2-yl)-4-vinylcyclohexyl)acrylaldehyde, 3,7,11-trimethyldodeca-1,3,5,8,10-pentaene, p-cymene, (1S,2R,4R,7E,11S)-4,8-dimethyl-12-methylidene-3,14-dioxatricyclo[9.3.0.02,4]tetradec-7-en-13-one, 3-[(3aS,4R,5R,8aR)-4-hydroxy-5,7-dimethyl-3-methylidene-2-oxo-4,5,8,8a-tetrahydro-3aH-cyclohepta[b]furan-6-yl]propyl acetate, 3,5,7-trihydroxy-2-(4-hydroxyphenyl)chromen-4-one, 2-(3,4-dihydroxyphenyl)-5,7-dihydroxychromen-4-one, 5,7-dihydroxy-2-(4-hydroxyphenyl)-3,6-dimethoxychromen-4-one, 2-(3,4-dihydroxyphenyl)-5,7-dihydroxy-3-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one, 1-(2-hydroxy-4-methoxyphenyl)-3-(4-hydroxyphenyl)propan-1-one, 3-(4-hydroxyphenyl)-1-[4-hydroxy-2-[3,4,5-trihydroxy-6-(hydroxymethyl)oxan-2-yl]oxyphenyl]propan-1-one, 3-(4-hydroxyphenyl)-1-[4-methoxy-2-[3,4,5-trihydroxy-6-(hydroxymethyl)oxan-2-yl]oxyphenyl]propan-1-one, 3-(4-hydroxyphenyl)-1-[2-hydroxy-4-[3,4,5-trihydroxy-6-(hydroxymethyl)oxan-2-yl]oxyphenyl]propan-1-one, (2S,3S,4S,5R,6S)-6-[5,7-dihydroxy-2-(4-hydroxyphenyl)-4-oxochromen-3-yl]oxy-3,4,5-trihydroxyoxane-2-carboxylic acid, 7-[(2S,3R,4S,5S,6R)-4,5-dihydroxy-6-(hydroxymethyl)-3-[(2S,3R,4R,5R,6S)-3,4,5-trihydroxy-6-methyloxan-2-yl]oxyoxan-2-yl]oxy-3,5-dihydroxy-2-(4-hydroxyphenyl)chromen-4-one, 3-[(2S,3R,4S,5S,6R)-4,5-dihydroxy-6-(hydroxymethyl)-3-[(2S,3R,4R,5R,6S)-3,4,5-trihydroxy-6-methyloxan-2-yl]oxyoxan-2-yl]oxy-5,7-dihydroxy-2-(4-hydroxyphenyl)chromen-4-one, 3-[(2S,5R)-3,5-dihydroxy-6-(hydroxymethyl)-4-[(2S,3S,5R)-3,4,5-trihydroxy-6-methyloxan-2-yl]oxyoxan-2-yl]oxy-5,7-dihydroxy-2-(4-hydroxyphenyl)chromen-4-one, [(2R,3S,4S,5R,6S)-6-[3,5-dihydroxy-2-(4-hydroxy-3-methoxyphenyl)-8-methoxy-4-oxochromen-7-yl]oxy-3,4-dihydroxy-5-[(2S,3R,4R,5R,6S)-3,4,5-trihydroxy-6-methyloxan-2-yl]oxyoxan-2-yl]methyl acetate, 5,7-dihydroxy-2-(4-hydroxyphenyl)-3-[(2S,3R,4S,5S)-3,4,5-trihydroxyoxan-2-yl]oxychromen-4-one, 3-[(2S,3R,4R,5S)-3,4-dihydroxy-5-(hydroxymethyl)oxolan-2-yl]oxy-5,7-dihydroxy-2-(4-hydroxyphenyl)chromen-4-one, (2S,3S,4S,5R,6S)-6-[4-(5,7-dihydroxy-3,6-dimethoxy-4-oxochromen-2-yl)-2-hydroxyphenoxy]-3,4,5-trihydroxyoxane-2-carboxylic acid, 2-(3,4-dihydroxyphenyl)-3,5,7-trihydroxychromen-4-one, 1,6-dihydroxy-3-methyl-8-[(2S,3R,4S,5S,6R)-3,4,5-trihydroxy-6-(hydroxymethyl)oxan-2-yl]oxyanthracene-9,10-dione, 1,3,8-trihydroxy-6-methylanthracene-9,10-dione, (E)-3-(4-hydroxyphenyl)-N-[4-[[(E)-3-(4-hydroxyphenyl)prop-2-enoyl]-[3-[[(E)-3-(4-hydroxyphenyl)prop-2-enoyl]amino]propyl]amino]butyl]prop-2-enamide, 5,7-dihydroxy-6-methoxy-2-(4-methoxyphenyl)chromen-4-one, 2-(3,4-dihydroxyphenyl)-5,7-dihydroxy-3-[(2S,3R,4S,5S,6R)-3,4,5-trihydroxy-6-(hydroxymethyl)oxan-2-yl]oxychromen-4-one, 2-(3,4-dihydroxyphenyl)-5,7-dihydroxy-6-methoxychromen-4-one, (E)-3-(4-hydroxyphenyl)-N-[3-[[(E)-3-(4-hydroxyphenyl)prop-2-enoyl]-[4-[[(E)-3-(4-hydroxyphenyl)prop-2-enoyl]-[3-[[(E)-3-(4-hydroxyphenyl)prop-2-enoyl]amino]propyl]amino]butyl]amino]propyl]prop-2-enamide, (1S,3R,4R,5R)-3,4-bis[[(E)-3-(3,4-dihydroxyphenyl)prop-2-enoyl]oxy]-1,5-dihydroxycyclohexane-1-carboxylic acid, and 5,7-dihydroxy-2-(4-hydroxyphenyl)chromen-4-one. In some embodiments, the oral composition is formulated as a dietary supplement, food ingredient or additive, a medical food, nutraceutical or pharmaceutical composition. In some embodiments, the oral composition is in a dosage form configured as one of a soft-gel capsule, a hard capsule, liquid, powder, and a tablet. In some embodiments, the oral composition comprises 10% to 99% w/w of the plant extract. In some embodiments, the plant extract further comprises a preservative. In some embodiments, the plant extract further comprises a sweetener. In some embodiments, the plant extract further comprises a coloring agent. In some embodiments, the plant extract further comprises an additive. In some embodiments, the plant extract further comprises a flavoring agent.

Some aspects of the disclosure relate to a method for improving, restoring, modulating, or maintaining sleep in a subject in need thereof. In some embodiments, the method includes administering a therapeutically effect amount of an oral composition as described herein. Some aspects of the disclosure relate to a method of treating a sleeping disorder in a subject in need thereof. In some embodiments, the method includes administering a therapeutically effect amount of an oral composition as described herein. In some embodiments, the plant extract includes an orexin receptor 2 antagonist. In some embodiments, the plant extract includes an orexin receptor 1 antagonist. In some embodiments, the plant extract further provides a decrease in calcium influx. In some embodiments, the plant extract further provides a decrease in pERK1/2 phosphorylation. In some embodiments, improving sleep provides a subject a full night's sleep. In some embodiments, improving sleep provides a subject a restful night's sleep. In some embodiments, modulating sleep provides a subject an increase in Slow-Wave-Sleep 1-4. In some embodiments, modulating sleep provides a subject an increase in REM sleep. In some embodiments, sleep disorder is selected from the group consisting of insomnias, sleep related breathing disorders, central disorders of hypersomnolence, circadian rhythm sleep-wake disorders, parasomnias, and sleep related movement disorders. In some embodiments, the sleep disorder is selected from the group consisting of insomnia, narcolepsy, hypersomnia, sleep apnoea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, and sleeping sickness.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1 illustrates a graph depicting in vitro bioactivity of compound 20 against orexin receptor 2.

FIG. 2 illustrates a graph depicting in vitro bioactivity of compound 21 against orexin receptor 2.

FIG. 3 illustrates a graph depicting in vitro bioactivity of compound 22 against orexin receptor 2.

FIG. 4 illustrates a graph depicting in vitro bioactivity of compound 23 against orexin receptor 2.

FIG. 5 illustrates a graph depicting in vitro bioactivity of compound 24 against orexin receptor 2.

FIG. 6 illustrates a graph depicting in vitro bioactivity of compound 25 against orexin receptor 2.

FIG. 7 illustrates a graph depicting in vitro bioactivity of compound 26 against orexin receptor 2.

FIG. 8 illustrates a graph depicting in vitro bioactivity of compound 27 against orexin receptor 2.

FIG. 9 illustrates a graph depicting in vitro bioactivity of compound 28 against orexin receptor 2.

FIG. 10 illustrates a graph depicting in vitro bioactivity of compound 29 against orexin receptor 2.

FIG. 11 illustrates a graph depicting in vitro bioactivity of compound 30 against orexin receptor 2.

FIG. 12 illustrates a graph depicting in vitro bioactivity of compounds 31-38 against orexin receptors (Orx 1 and Orx2).

### DETAILED DESCRIPTION OF THE DISCLOSURE

This disclosure provides, among other things, the discovery of compounds, compositions, and methods for improving, restoring, modulating, or maintaining sleep in a subject. In aspects, the compositions include one or more compounds as described herein and a carrier. In some aspects, methods include orally administering one or more compounds as described herein or a composition as described herein, thereby improving, restoring, modulating, or maintaining sleep in a subject. In some embodiments, the oral composition may, for example, promote the maintenance of sleep leading to a restful night's sleep, further improving the quality of a person's sleep. The oral compositions described herein employed in various embodiments of the present disclosure have been shown to be antagonists to certain receptors that may relate to the induction and modulation of sleep.

Moreover, the oral compositions disclosed herein may also be useful in modulating satiation or disorders associated with satiation.

### Definitions

Unless expressly defined otherwise, technical and/or scientific terms used herein have the same meaning as is commonly understood by one of ordinary skill in the art. In the event that there are a plurality of definitions for a term herein, those in this section prevail unless stated otherwise. As used in the specification and the appended claims, the singular forms “a,” “an,” and “the” include plural referents unless the context clearly dictates otherwise. The use of either the conjunction “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Compounds disclosed herein having at least one chiral center they may exist as a racemate or as each enantiomer, and may exist as enantiomeric-enriched mixtures of the enantiomers. It should be noted that all such isomers and mixtures thereof are included in the scope of the present invention. Furthermore, the crystalline forms for the compounds disclosed herein may exist as alternative polymorphs. Such polymorphs are included in one embodiment of the present invention. In addition, some of the compounds of the present invention may form solvates with water (i.e., hydrates) or common organic solvents. Such solvates are included in one embodiment of the present invention.

The term “pharmaceutically acceptable salt,” as used herein, refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

The term “pharmaceutically acceptable carrier” or “pharmaceutically acceptable excipient,” as used herein, includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “halogen,” as used herein, means any one of the radio-stable atoms of column 7 of the Periodic Table of the Elements, e.g., fluorine, chlorine, bromine, or iodine.

Any amine, hydroxyl, or carboxyl side chain on the compounds disclosed herein can be esterified or amidified. The procedures and specific groups to be used to achieve this end are known to those of skill in the art and can readily be found in reference sources such as Greene and Wuts, Protective Groups in Organic Synthesis, 3^{rd} Ed., John Wiley & Sons, New York, NY, 1999, which is incorporated herein in its entirety.

As used herein, “Cₐ to C_{b}” or “Cₐ-C_{b}” in which “a” and “b” are integers refer to the number of carbon atoms in an alkyl, alkenyl or alkynyl group, or the number of carbon atoms in the ring of a cycloalkyl, aryl, heteroaryl or heterocyclyl group. That is, the alkyl, alkenyl, alkynyl, ring of the cycloalkyl, ring of the aryl, ring of the heteroaryl or ring of the heterocyclyl can contain from “a” to “b”, inclusive, carbon atoms. Thus, for example, a “C₁ to C₄ alkyl” group or a “C₁-C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)- and (CH₃)₃C—. Likewise, for example, cycloalkyl group may contain from “a” to “b”, inclusive, total atoms, such as a C₃-C₈ cycloalkyl group, 3 to 8 carbon atoms in the ring(s). If no “a” and “b” are designated with regard to an alkyl, cycloalkyl, or cycloalkenyl, the broadest range described in these definitions is to be assumed. Similarly, a “4 to 7 membered heterocyclyl” group refers to all heterocyclyl groups with 4 to 7 total ring atoms, for example, azetidine, oxetane, oxazoline, pyrrolidine, piperidine, piperazine, morpholine, and the like. As used herein, the term “C₁-C₆” includes C₁, C₂, C₃, C₄, C₅ and C₆, and a range defined by any of the two preceding numbers. For example, C₁-C₆ alkyl includes C₁, C₂, C₃, C₄, C₅ and C₆ alkyl, C₂-C₆ alkyl, C₁-C₃ alkyl, etc. Similarly, C₃-C₈ carbocyclyl or cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl. As another example, 3 to 10 membered heterocyclyl includes 3, 4, 5, 6, 7, 8, 9, or 10 ring atoms, or a range defined by any of the two preceding numbers, such as 4 to 6 membered or 5 to 7 membered heterocyclyl.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substituent may be substituted with one of the above substituents.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups may range from C₃ to C₁₀, in other embodiments it may range from C₃ to C₆. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated. When substituted, substituents on a cycloalkyl group may form an aromatic ring fused to the cycloalkyl group, including an aryl and a heteroaryl.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, the term “amino” refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on an aryl group may form a non-aromatic ring fused to the aryl group, including a cycloalkyl, cycloalkenyl, cycloalkynyl, and heterocyclyl.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. When substituted, substituents on a heteroaryl group may form a non-aromatic ring fused to the aryl group, including a cycloalkyl, cycloalkenyl, cycloalkynyl, and heterocyclyl.

As used herein, “heterocyclyl” refers to a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

The terms “purified,” “substantially purified,” and “isolated” as used herein, refer to compounds disclosed herein being free of other, dissimilar compounds with which the compounds of the invention are normally associated in their natural state, so that the compounds of the invention comprise at least 0.5%, 1%, 5%, 10%, or 20%, and most preferably at least 50% or 75% of the mass, by weight, of a given sample.

Substituted groups are based upon or derived from the unsubstituted parent group in which there has been an exchange of one or more hydrogen atoms for another atom or group. Unless otherwise indicated, when a group is deemed to be “substituted,” or “optionally substituted,” the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

The term “subject,” as used herein, refers to a human or a non-human mammal, e.g., a dog, a cat, a mouse, a rat, a cow, a sheep, a pig, a goat, a non-human primate or a bird, e.g., a chicken, as well as any other vertebrate or invertebrate.

It is to be understood that where compounds disclosed herein have unfilled valencies, then the valencies are to be filled with hydrogens and/or deuteriums.

The term “about,” as used herein, refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. When a value is preceded by the term about, the component is not intended to be limited strictly to that value, but it is intended to include amounts that vary from the value.

### Compounds

In some aspects, the disclosure provides for a compound of Formula (I).

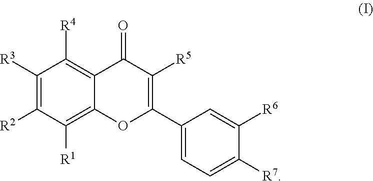

In some embodiments, Formula (I) is a salt as described herein. In some embodiments, the salt is suitable for oral consumption. In some embodiments, the salt is a pharmaceutically acceptable salt.

In some embodiments, Formula (I) is represented by Formula (IA)

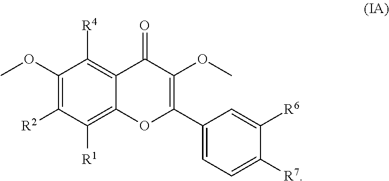

In some embodiments, Formula (I) is represented by Formula (IB)

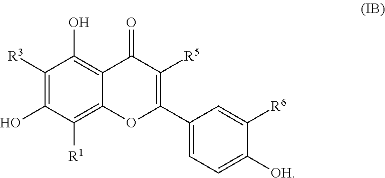

In some aspects, the disclosure provides for a compound of Formula (II).

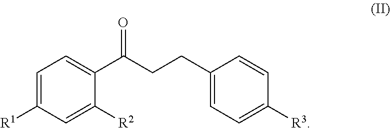

In some embodiments, Formula (II) is a salt as described herein. In some embodiments, the salt is suitable for oral consumption. In some embodiments, the salt is a pharmaceutically acceptable salt.

In some aspects, the disclosure provides for a compound of Formula (III).

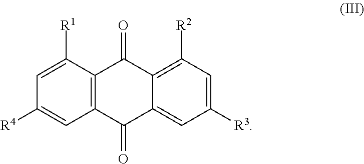

In some embodiments, Formula (III) is a salt as described herein. In some embodiments, the salt is suitable for oral consumption. In some embodiments, the salt is a pharmaceutically acceptable salt.

In some embodiments of the compounds of Formula (I), (IA), (IB), (II), or (III), R¹ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I), (IA), (II), or (III), R² may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I), (IB), (II), or (III), R³ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I) or (IA), R⁴ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I) or (IB), R⁵ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I) or (IB), R⁶ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I), R⁷ may be selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L.

In some embodiments of the compounds of Formula (I), (IA), (IB), (II), or (III), L is —Z¹-Z². In some embodiments of the compounds of Formula (I), (IA), (IB), (II) or (IIII), L is —Z¹-Z²—Z³. In some embodiments, L is a monosaccharide. In some embodiments, L is a polysaccharide. In some embodiments of the compounds of Formula (I), (IA), (IB), (II), or (III), at least one of R¹-R⁷ is L. In some embodiments of the compounds of (I), (IA), (IB), (II), or (III), at least one of R¹-R⁷ is L and L is —Z¹-Z². In some embodiments of the compounds of Formula (I), (IA), (IB), (II), or (III), only one of R¹-R⁷ is L and L is —Z¹-Z². In some embodiments of the compounds of Formula (I), (IA), (IB), (II), or (III), only one of R¹-R⁷ is an optionally substituted C₃-C₈ heterocyclyl.

In some embodiments of the compounds of Formula (I), (IA), or (II), Z₁ is —CH₂—, —O—, —NH—, optionally substituted C₃-C₈ cycloalkyl, optionally substituted C₆ to C₁₀ aryl, optionally substituted —C₃-C₈ heterocyclyl, or optionally substituted C₃ to C₁₀ heteroaryl. In some embodiments, Z1 is optionally substituted —C₃-C₈ heterocyclyl.

In some embodiments of the compounds of Formula (I), (IA), (IB), (II), or (III), Z² is hydrogen, deuterium, halo, —CH₂—, —O—, —CO₂H, —CO₂CHCH—, optionally substituted —C₃-C₈ cycloalkyl, optionally substituted —C₆-C₁₀ aryl, optionally substituted —C₃-C₈ heterocyclyl, or optionally substituted —C₃-C₁₀ heteroaryl. In some embodiments, Z² is optionally substituted —C₃-C₈ heterocyclyl.

In some embodiments of the compounds of Formula (I), (IA), (IB), (II), or (III), Z³ is hydrogen, deuterium, halo, —CH₂—, —O—, optionally substituted —C₃-C₈ cycloalkyl, optionally substituted —C₆-C₁₀ aryl, optionally substituted —C₃-C₈ heterocyclyl, or optionally substituted —C₃-C₁₀ heteroaryl. In some embodiments, Z² is optionally substituted —C₃-C₈ heterocyclyl.

In some embodiments, the optionally substituted C₃-C₈ heterocyclyl is

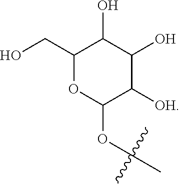 [SMILES: *OC1OC(CO)C(O)C(O)C1O]

In some embodiments, the optionally substituted C₃-C₈ heterocyclyl is a monosaccharide. In some embodiments, the optionally substituted C₃-C₈ heterocyclyl is selected from the group consisting of glucose, mannose, fructose, and xylose, and rhamnose. In some embodiments, the optionally substituted C₃-C₈ heterocyclyl is an enantiomer, and may be D-glucose. In some embodiments, the optionally substituted C₃-C₈ heterocyclyl is

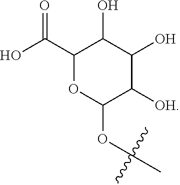 [SMILES: *OC1OC(C(=O)O)C(O)C(O)C1O]

In some embodiments, the optionally substituted C₃-C₈ heterocyclyl is

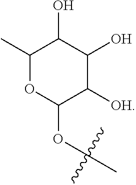 [SMILES: *OC1OC(C)C(O)C(O)C1O]

In some embodiments, the optionally substituted C₃-C₈ heterocyclyl is

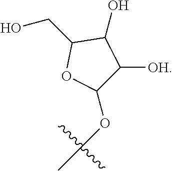 [SMILES: *OC1OC(CO)C(O)C1O]

For example, in some embodiments, a compound according to Formula (I), (IA), (IB), (II), or (III) may be structurally similar compound as compared to a naturally occurring source, but may, for example, not be a naturally occurring compound. In some embodiments, a compound according to Formula (I), (IA), (IB), (II), or (III) may be chemically modified from a naturally occurring compound. For example, a compound according to Formula (I), (IA), (IB), (II), or (III) may be deuterated at one or more locations to replace a hydrogen, and is not itself a naturally occurring compound.

In some embodiments of the compounds of Formula (I), (IA), (IB), (II), or (III), are selected from Compounds of Table A, salt thereof, or pharmaceutically acceptable salts thereof. Table A depicts compounds according to the disclosure, and may include isomers and stereoisomers of the compounds portrayed. For example, compounds of Table A may depict cis and trans isomers by depicting a crossed double bond:

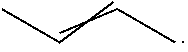

TABLE A

| Cmpd | |
|---|---|
| ID | Structure |
| 1 | 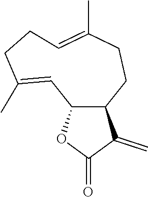 |
| 2 | 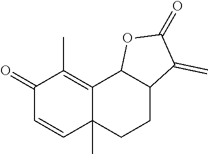 [SMILES: C=C1C(=O)OC2C3=C(C)C(=O)C=CC3(C)CCC12] |
| 3 | 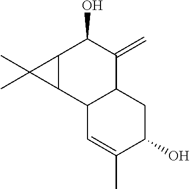 [SMILES: C=C1C2C[C@H](O)C(C)=CC2C2C([C@H]1O)C2(C)C] |
| 4 | 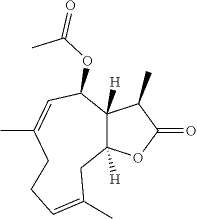 [SMILES: CC(=O)O[C@@H]1/C=C(/C)CC/C=C(/C)C[C@@H]2OC(=O)[C@H](C)[C@H]21] |
| 5 | 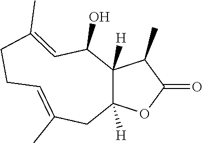 [SMILES: C/C1=C\[C@@H](O)[C@H]2[C@H](C/C(C)=C/CC1)OC(=O)[C@@H]2C] |
| 6 | 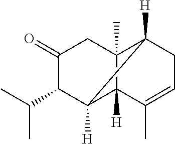 [SMILES: CC1=CC[C@@H]2[C@H]3[C@@H]1[C@@]2(C)CC(=O)[C@H]3C(C)C] |
| 7 | 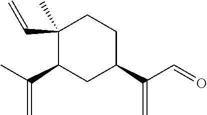 [SMILES: C=C[C@@]1(C)CC[C@@H](C(=C)C=O)C[C@H]1C(=C)C] |
| 8 | 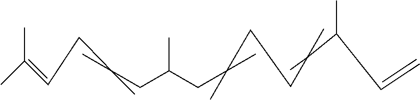 [SMILES: C=CC(C)C(C)(C)CCC(C)(C)CC(C)CC(C)(C)CC=C(C)C] |
| 9 | 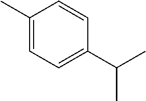 |
| 10 | 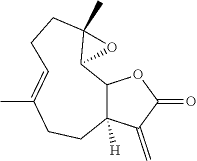 [SMILES: C=C1C(=O)OC2[C@@H]1CC/C(C)=C/CC[C@]1(C)O[C@@H]21] |
| 11 | 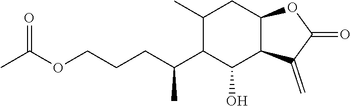 [SMILES: C=C1C(=O)O[C@@H]2CC(C)C([C@@H](C)CCCOC(C)=O)[C@@H](O)[C@H]12] |
| 12 | 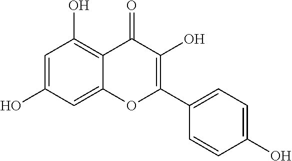 [SMILES: O=c1c(O)c(-c2ccc(O)cc2)oc2cc(O)cc(O)c12] |
| 13 | 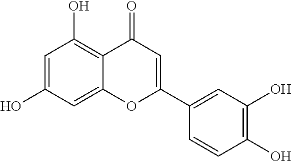 [SMILES: O=c1cc(-c2ccc(O)c(O)c2)oc2cc(O)cc(O)c12] |
| 14 | 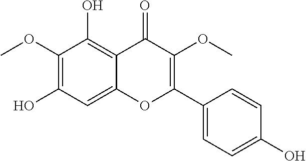 [SMILES: COc1c(O)cc2oc(-c3ccc(O)cc3)c(OC)c(=O)c2c1O] |
| 15 | 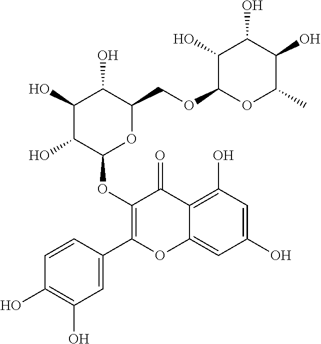 [SMILES: C[C@@H]1O[C@@H](OC[C@H]2O[C@@H](Oc3c(-c4ccc(O)c(O)c4)oc4cc(O)cc(O)c4c3=O)[C@H](O)[C@@H](O)[C@@H]2O)[C@H](O)[C@H](O)[C@H]1O] |
| 16 | 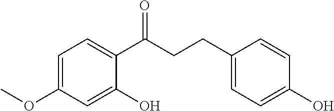 [SMILES: COc1ccc(C(=O)CCc2ccc(O)cc2)c(O)c1] |
| 17 | 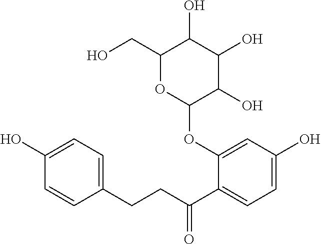 [SMILES: O=C(CCc1ccc(O)cc1)c1ccc(O)cc1OC1OC(CO)C(O)C(O)C1O] |
| 18 | 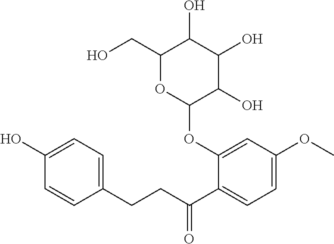 [SMILES: COc1ccc(C(=O)CCc2ccc(O)cc2)c(OC2OC(CO)C(O)C(O)C2O)c1] |
| 19 | 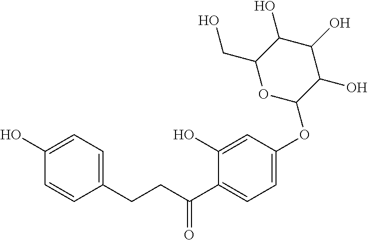 [SMILES: O=C(CCc1ccc(O)cc1)c1ccc(OC2OC(CO)C(O)C(O)C2O)cc1O] |
| 20 | 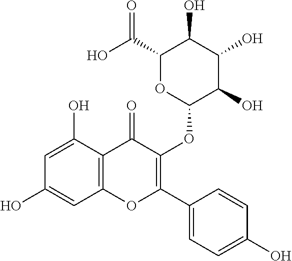 [SMILES: O=C(O)[C@H]1O[C@@H](Oc2c(-c3ccc(O)cc3)oc3cc(O)cc(O)c3c2=O)[C@H](O)[C@@H](O)[C@@H]1O] |
| 21 | 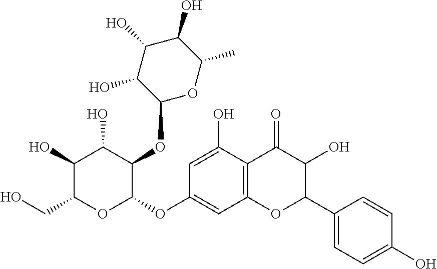 [SMILES: C[C@@H]1O[C@@H](O[C@H]2[C@H](Oc3cc(O)c4c(c3)OC(c3ccc(O)cc3)C(O)C4=O)O[C@H](CO)[C@@H](O)[C@@H]2O)[C@H](O)[C@H](O)[C@H]1O] |
| 22 | 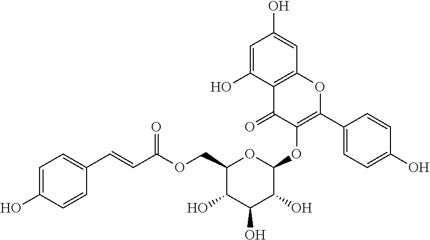 [SMILES: O=C(/C=C/c1ccc(O)cc1)OC[C@H]1O[C@@H](Oc2c(-c3ccc(O)cc3)oc3cc(O)cc(O)c3c2=O)[C@H](O)[C@@H](O)[C@@H]1O] |
| 23 | 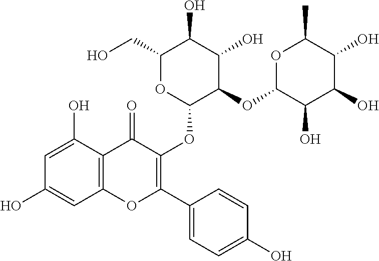 [SMILES: C[C@@H]1O[C@@H](O[C@H]2[C@H](Oc3c(-c4ccc(O)cc4)oc4cc(O)cc(O)c4c3=O)O[C@H](CO)[C@@H](O)[C@@H]2O)[C@H](O)[C@H](O)[C@H]1O] |
| 24 | 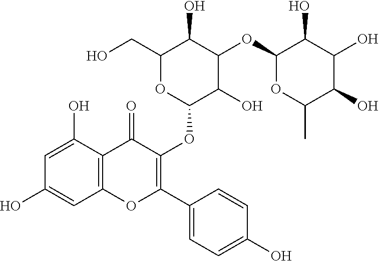 [SMILES: CC1O[C@@H](OC2C(O)[C@H](Oc3c(-c4ccc(O)cc4)oc4cc(O)cc(O)c4c3=O)OC(CO)[C@H]2O)[C@@H](O)C(O)[C@H]1O] |
| 25 | 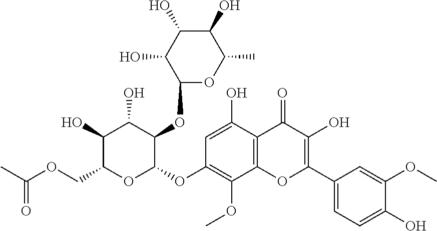 [SMILES: COc1cc(-c2oc3c(OC)c(O[C@@H]4O[C@H](COC(C)=O)[C@@H](O)[C@H](O)[C@H]4O[C@@H]4O[C@@H](C)[C@H](O)[C@@H](O)[C@H]4O)cc(O)c3c(=O)c2O)ccc1O] |
| 26 | 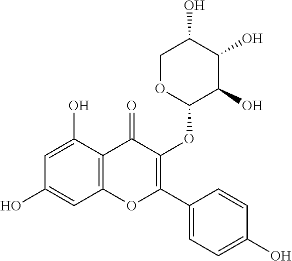 [SMILES: O=c1c(O[C@@H]2OC[C@H](O)[C@H](O)[C@H]2O)c(-c2ccc(O)cc2)oc2cc(O)cc(O)c12] |
| 27 | 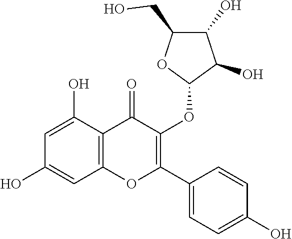 [SMILES: O=c1c(O[C@@H]2O[C@@H](CO)[C@H](O)[C@H]2O)c(-c2ccc(O)cc2)oc2cc(O)cc(O)c12] |
| 28 | 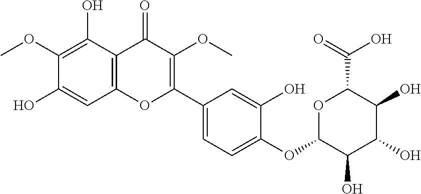 [SMILES: COc1c(O)cc2oc(-c3ccc(O[C@@H]4O[C@H](C(=O)O)[C@@H](O)[C@H](O)[C@H]4O)c(O)c3)c(OC)c(=O)c2c1O] |
| 29 | 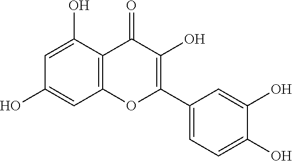 [SMILES: O=c1c(O)c(-c2ccc(O)c(O)c2)oc2cc(O)cc(O)c12] |
| 30 | 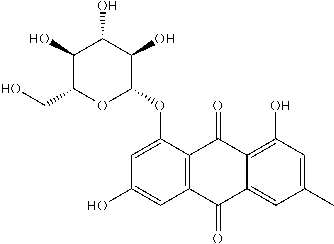 [SMILES: Cc1cc(O)c2c(c1)C(=O)c1cc(O)cc(O[C@@H]3O[C@H](CO)[C@@H](O)[C@H](O)[C@H]3O)c1C2=O] |
| 31 | 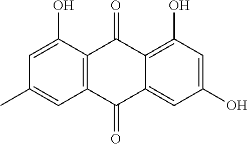 [SMILES: Cc1cc(O)c2c(c1)C(=O)c1cc(O)cc(O)c1C2=O] |
| 32 | 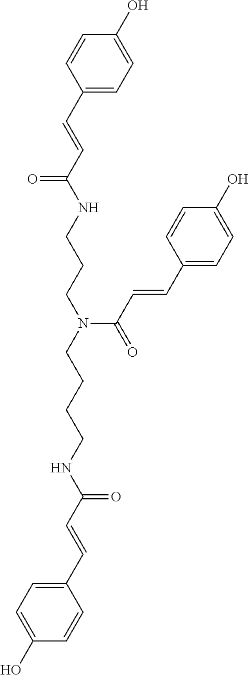 [SMILES: O=C(/C=C/c1ccc(O)cc1)NCCCCN(CCCNC(=O)/C=C/c1ccc(O)cc1)C(=O)/C=C/c1ccc(O)cc1] |
| 33 | 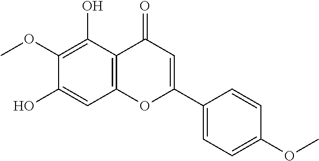 |
| 34 | 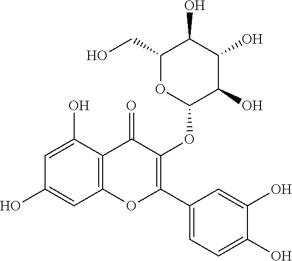 [SMILES: O=c1c(O[C@@H]2O[C@H](CO)[C@@H](O)[C@H](O)[C@H]2O)c(-c2ccc(O)c(O)c2)oc2cc(O)cc(O)c12] |
| 35 | 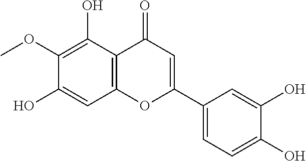 [SMILES: COc1c(O)cc2oc(-c3ccc(O)c(O)c3)cc(=O)c2c1O] |
| 36 | 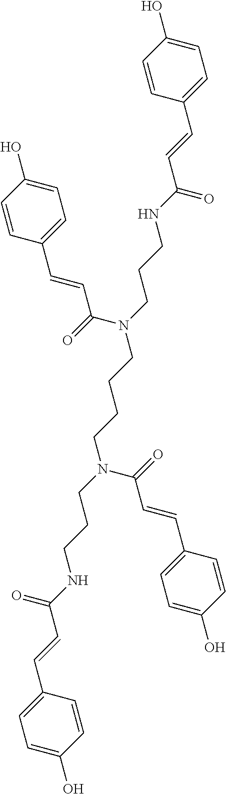 [SMILES: O=C(/C=C/c1ccc(O)cc1)NCCCN(CCCCN(CCCNC(=O)/C=C/c1ccc(O)cc1)C(=O)/C=C/c1ccc(O)cc1)C(=O)/C=C/c1ccc(O)cc1] |
| 37 | 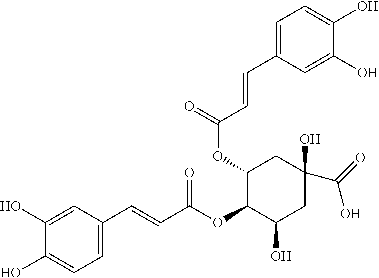 [SMILES: O=C(/C=C/c1ccc(O)c(O)c1)O[C@@H]1[C@H](O)C[C@@](O)(C(=O)O)C[C@H]1OC(=O)/C=C/c1ccc(O)c(O)c1] |
| 38 | 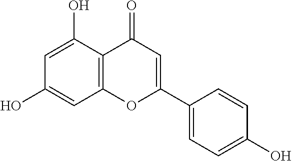 [SMILES: O=c1cc(-c2ccc(O)cc2)oc2cc(O)cc(O)c12] |

In some embodiments, the compound of Formula (I) does not include Compound 12, 13, 14, 15, 20, 21, 22, 23, 24, 25, 26, 27, 28, 29, 33, 34, 35, 38, or a combination thereof. In some embodiments, the compound of Formula (II) does not include Compound 16, 17, 18, 19, or a combination thereof. In some embodiments, the compound of Formula (III) does not include Compound 30, 31, or a combination thereof.

In some embodiments, the disclosed chemical structures may contain glucosides, or other structures where the sugar part is selected from the nonlimiting examples of glucose, fructose, galactose, mannose, xylose, rhamnose, sialic acid, and a sugar derivative. The metabolites of such structures may vary depending on the specific sugar/glucoside; however, a primary metabolic reaction for glucosides is hydrolysis. This process involves the breaking of the glycosidic bond between the glucose unit and the aglycone (the non-sugar part of the molecule). Enzymes called glucosidases, which are present in various organisms, facilitate this reaction. The hydrolysis yields a sugar component (glucose) and the aglycone. Accordingly, the disclosure provides for aglycones for any of the disclosed compounds listed in Table A. In some embodiments, the disclosure provides for substituted flavonoids, substituted flavanones, substituted flavones, substituted chalcones, and substituted anthraquinones. In some embodiments, a flavonoid core molecule may be produced from a chalcone by isomerization with the enzyme chalcone isomerase (CHI) for the generation of the flavanone group.

A salt of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like; or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic camphorsulfonic acid, acid, 4-toluenesulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition, it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns. Likewise, it is understood that, in any compound described, all tautomeric forms are also intended to be included.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the oral compositions, formulations, and methods described herein include the use of crystalline forms, amorphous phases, and/or pharmaceutically acceptable salts, solvates, hydrates, and conformers of compounds of some embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. In specific embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein. Other forms in which the compounds of some embodiments can be provided include amorphous forms, milled forms and nano-particulate forms.

Likewise, it is understood that the compounds described herein, such as compounds of some embodiments, include the compound in any of the forms described herein (e.g., pharmaceutically acceptable salts, prodrugs, crystalline forms, amorphous form, solvated forms, enantiomeric forms, tautomeric forms, and the like).

### Compositions

In aspects, disclosed herein are compositions comprising one or more compounds described herein and a carrier. In some embodiments, the one or more compounds are plant-derived. In some embodiments, the one or more compounds are derived from a cell. In some embodiments, the one or more compounds are synthetically produced. In some embodiments, the composition is an oral composition.

In some embodiments, the composition includes a plant extract including one or more compounds as described herein and a carrier. As used herein, an “extract” refers a composition containing a desired compound of interest which is separated from other substances present in the natural source material from which the oral composition was obtained. Plant extracts can be obtained from any plant tissue including a whole plant; plant part such as shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary); plant tissue (for example, vascular tissue, ground tissue, and the like); or cell (for example, guard cells, egg cells, and the like), and progeny and cultures or cell lines of the same. In some embodiments, the extract is generally recognized as safe for human consumption. Accordingly, in certain embodiments the extract is from an edible source. In this respect, the extract is an edible extract. Extracts can be prepared by freezing, grinding, macerating, pulverizing and/or fermenting the source material of interest, subjecting the source material to solvent extraction, and separating the insoluble material from soluble material. In this respect, an “extract” of the disclosure can be crude, fractionated, sub-fractionated, separated, isolated, enriched or purified, without being limited thereto.

In some embodiments, the plant extract may be a crude extract. As used herein, “crude” refers to compounds or molecules that have not been entirely separated from the components of the original composition in which it was present. In embodiments pertaining to fractions or sub-fractions, a molecule in crude extract may be subjected to partial separation to provide a less crude extract containing other substances. By comparison, the term “isolated” means that a compound or molecule is substantially enriched or purified with respect to the complex cellular milieu in which it naturally occurs, such as in a crude extract. When an isolated molecule is enriched or purified, the absolute level of purity is not critical and those skilled in the art can readily determine appropriate levels of purity according to the use to which the material is to be put. In some circumstances, the isolated molecule forms part of a composition (for example a more or less crude extract containing many other substances), which may for example contain other components. In other circumstances, the isolated molecule may be purified to essential homogeneity, for example as determined spectrophotometrically, by NMR or by chromatography (for example LC-MS).

Suitable solvents for preparing an extract include, e.g., n-pentane, hexane, butane, chloroform, dichloromethane, di-ethyl ether, acetonitrile, water, butanol, isopropanol, ethanol, methanol, glacial acetic acid, acetone, norflurane (HFA134a), ethyl acetate, dimethyl sulfoxide, heptafluoropropane (HFA227), and subcritical or supercritical fluids such as liquid carbon dioxide and water, or a combination thereof in any proportion. When solvents such as those listed above are used, the resultant extract typically contains non-specific lipid-soluble material. This can be removed by a variety of processes including “winterization”, which involves chilling to a specified temperature, typically −20° C. followed by filtration or centrifugation to remove waxy ballast, extraction with subcritical or supercritical carbon dioxide or non-polar solvents (e.g., hexane) and by distillation.

In some embodiments, the plant extract includes Eupatorium perfoliatum. In some embodiments, the plant extract includes a purified or crude extract of Eupatorium perfoliatum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 3′,4′,5,7-Tetrahydroxy-3-[α-L-rhamnopyranosyl-(1→6)-β-D-glucopyranosyloxy]flavone. In some embodiments, the plant extract includes 3,3′,4′,5,7-Pentahydroxyflavone. In some embodiments, the plant extract includes Compound 13. In some embodiments, the plant extract includes Compound 14.

In some embodiments, the plant extract includes Zingiber officinale. In some embodiments, the plant extract includes a purified or crude extract of Zingiber officinale. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Picea sitchensis. In some embodiments, the plant extract includes a purified or crude extract of Picea sitchensis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Viburnum davidii. In some embodiments, the plant extract includes a purified or crude extract of Viburnum davidii. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 1-(2-hydroxy-4-{[3,4,5-trihydroxy-6-(hydroxymethyl)oxan-2-yl]oxy}phenyl)-3-(4-hydroxyphenyl)propan-1-one. In some embodiments, the plant extract includes 3-(4-hydroxyphenyl)-1-[4-hydroxy-2-[3,4,5-trihydroxy-6-(hydroxymethyl)oxan-2-yl]oxyphenyl]propan-1-one. In some embodiments, the plant extract includes 4′-O-Methyldavidioside. In some embodiments, the plant extract includes Compound 16. In some embodiments, the plant extract includes Compound 17. In some embodiments, the plant extract includes Compound 19.

In some embodiments, the plant extract includes Arnica montana. In some embodiments, the plant extract includes a purified or crude extract of Arnica montana. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 3,4′,5,7-Tetrahydroxyflavone. In some embodiments, the plant extract includes Compound 12.

In some embodiments, the plant extract includes Helianthus annuus. In some embodiments, the plant extract includes a purified or crude extract of Helianthus annuus. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Lactuca sativa. In some embodiments, the plant extract includes a purified or crude extract of Lactuca sativa. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Barnadesia spinosa. In some embodiments, the plant extract includes a purified or crude extract of Barnadesia spinosa. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Dolomiaea souliei. In some embodiments, the plant extract includes a purified or crude extract of Dolomiaea souliei. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Saussurea costus. In some embodiments, the plant extract includes a purified or crude extract of Saussurea costus. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Artemisia annua. In some embodiments, the plant extract includes a purified or crude extract of Artemisia annua. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Inula helenium. In some embodiments, the plant extract includes a purified or crude extract of Inula helenium. In some embodiments, the extract may be derived directly from whole seeds. In some embodiments, the extract may be derived directly from leaves. In some embodiments, the extract may be derived directly from whole seeds. In some embodiments, the extract may be derived directly from flowers. I In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Laurus nobilis. In some embodiments, the plant extract includes a purified or crude extract of Laurus nobilis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Momodica cochinchinensis. In some embodiments, the plant extract includes a purified or crude extract of Momodica cochinchinensis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Pogostemon cablin. In some embodiments, the plant extract includes a purified or crude extract of Pogostemon cablin. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Cichorium intybus. In some embodiments, the plant extract includes a purified or crude extract of Cichorium intybus. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Solidago Canadensis. In some embodiments, the plant extract includes a purified or crude extract of Solidago Canadensis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Horeum vulgare. In some embodiments, the plant extract includes a purified or crude extract of Horeum vulgare. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Tricum aestivum. In some embodiments, the plant extract includes a purified or crude extract of Tricum aestivum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Silene conica. In some embodiments, the plant extract includes a purified or crude extract of Silene conica. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Alloteropsis semialata. In some embodiments, the plant extract includes a purified or crude extract of Alloteropsis semialata. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Paris polyphylla. In some embodiments, the plant extract includes a purified or crude extract of Paris polyphylla. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Camella sinensis. In some embodiments, the plant extract includes a purified or crude extract of Camella sinensis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Hordeum vulgare. In some embodiments, the plant extract includes a purified or crude extract of Hordeum vulgare. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Setaria italic. In some embodiments, the plant extract includes a purified or crude extract of Setaria italic. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Zea mays. In some embodiments, the plant extract includes a purified or crude extract of Zea mays. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the seeds, leaves and/or flowers may be ground into a powder, which is then heated to a temperature selected from a range of about 30 to 100° C., to remove oils prior to proceeding with an extraction step.

In some embodiments, the plant extract includes Arabidopsis thaliana. In some embodiments, the plant extract includes a purified or crude extract of Arabidopsis thaliana. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Brachypodium distachyon. In some embodiments, the plant extract includes a purified or crude extract of Brachypodium distachyon. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Grindelia integrifolia. In some embodiments, the plant extract includes a purified or crude extract of Grindelia integrifolia. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Lobelia chinesis. In some embodiments, the plant extract includes a purified or crude extract of Lobelia chinesis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Arachis hypogaea. In some embodiments, the plant extract includes a purified or crude extract of Arachis hypogaea. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Nepenthes khasiana. In some embodiments, the plant extract includes a purified or crude extract of Nepenthes khasiana. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Selaginella mollendorffii. In some embodiments, the plant extract includes a purified or crude extract of Selaginella mollendorffii. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Selaginella mollendorffii. In some embodiments, the plant extract includes a purified or crude extract of Selaginella mollendorffii. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Taxus×media. In some embodiments, the plant extract includes a purified or crude extract of Taxus×media. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Acorus gramieus. In some embodiments, the plant extract includes a purified or crude extract of Acorus gramieus. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Andrographis paniculata. In some embodiments, the plant extract includes a purified or crude extract of Andrographis paniculata. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Artemisia maritima. In some embodiments, the plant extract includes a purified or crude extract of Artemisia maritima. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Bletilla striata. In some embodiments, the plant extract includes a purified or crude extract of Bletilla striata. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Fallopia multflora. In some embodiments, the plant extract includes a purified or crude extract of Fallopia multflora. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Gossypium hirsutum. In some embodiments, the plant extract includes a purified or crude extract of Gossypium hirsutum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Hordeum spontaneum. In some embodiments, the plant extract includes a purified or crude extract of Hordeum spontaneum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Hypericum perforatum. In some embodiments, the plant extract includes a purified or crude extract of Hypericum perforatum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Musa acuminate. In some embodiments, the plant extract includes a purified or crude extract of Musa acuminate. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Oryza rufipogon. In some embodiments, the oral composition includes an extract of Oryza rufipogon. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Oryza sativa. In some embodiments, the plant extract includes a purified or crude extract of Oryza sativa. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Quercus suber. In some embodiments, the plant extract includes a purified or crude extract of Quercus suber. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Solanum tuberosum. In some embodiments, the plant extract includes a purified or crude extract of Solanum tuberosum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Valeriana officinallis. In some embodiments, the plant extract includes a purified or crude extract of Valeriana officinallis. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Vitis vinifera. In some embodiments, the plant extract includes a purified or crude extract of Vitis vinifera. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Polygonatum odoratum. In some embodiments, the plant extract includes a purified or crude extract of Polygonatum odoratum. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Glycine max. In some embodiments, the plant extract includes a purified or crude extract of Glycine max. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Panicum virgatum cv. Trailblazer. In some embodiments, the plant extract includes a purified or crude extract of Panicum virgatum cv. Trailblazer. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Populus trichorarpa. In some embodiments, the plant extract includes a purified or crude extract of Populus trichorarpa. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step.

In some embodiments, the plant extract includes Eruca vesicaria. In some embodiments, the plant extract includes a purified or crude extract of Eruca vesicaria. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes [(2R,3S,4S,5R,6S)-6-{[2-(3,4-dihydroxyphenyl)-5,7-dihydroxy-4-oxo-4H-chromen-3-yl]oxy}-3,4,5-trihydroxyoxan-2-yl]methyl (2E)-3-(4-hydroxyphenyl)prop-2-enoate. In some embodiments, the plant extract includes Compound 22.

In some embodiments, the plant extract includes Gynura procumbens. In some embodiments, the plant extract includes a purified or crude extract of Gynura procumbens. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 7-[(2S,3R,4S,5S,6R)-4,5-Dihydroxy-6-(hydroxymethyl)-3-[(2S,3R,4R,5R,6S)-3,4,5-trihydroxy-6-methyloxan-2-yl]oxyoxan-2-yl]oxy-3,5-dihydroxy-2-(4-hydroxyphenyl)chromen-4-one. In some embodiments, the plant extract includes Compound 21.

In some embodiments, the plant extract includes Clausena excavata. In some embodiments, the plant extract includes a purified or crude extract of Clausena excavata. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 3-[(2S,5R)-3,5-dihydroxy-6-(hydroxymethyl)-4-[(2S,3S,5R)-3,4,5-trihydroxy-6-methyloxan-2-yl]oxyoxan-2-yl]oxy-5,7-dihydroxy-2-(4-hydroxyphenyl)chromen-4-one. In some embodiments, the plant extract includes Compound 20. In some embodiments, the plant extract includes Compound 24.

In some embodiments, the plant extract includes Psidium guajava. In some embodiments, the plant extract includes a purified or crude extract of Psidium guajava. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 3-[(2S,3R,4R,5S)-3,4-dihydroxy-5-(hydroxymethyl)oxolan-2-yl]oxy-5,7-dihydroxy-2-(4-hydroxyphenyl)chromen-4-one. In some embodiments, the plant extract includes Compound 27.

In some embodiments, the plant extract includes Senna alexandrina. In some embodiments, the plant extract includes a purified or crude extract of Senna alexandrina. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 3-(((2R,3S,4R,5R,6S)-4,5-dihydroxy-6-(hydroxymethyl)-3-(((2S,3R,4R,5R,6S)-3,4,5-trihydroxy-6-methyltetrahydro-2H-pyran-2-yl)oxy)tetrahydro-2H-pyran-2-yl)oxy)-5,7-dihydroxy-2-(4-hydroxyphenyl)-4H-chromen-4-one. In some embodiments, the plant extract includes Compound 23. In some embodiments, the plant extract includes Compound 26.

In some embodiments, the plant extract includes Tanacetum parthenium. In some embodiments, the plant extract includes a purified or crude extract of Tanacetum parthenium. In some embodiments, the extract may be derived directly from whole seeds. In some embodiments, the extract may be derived directly from leaves. In some embodiments, the extract may be derived directly from whole seeds. In some embodiments, the extract may be derived directly from flowers. In some embodiments, the extract may be derived directly from roots. In some embodiments, the seeds, leaves, flowers and/or roots may be ground into a powder, which is then heated to a temperature selected from a range of about 30 to 100° C., to remove oils prior to proceeding with an extraction step. In some embodiments, the plant extract includes 1,6-dihydroxy-3-methyl-8-[(2S,3R,4S,5S,6R)-3,4,5-trihydroxy-6-(hydroxymethyl)oxan-2-yl]oxyanthracene-9,10-dione. In some embodiments, the plant extract includes 3,6-Dimethoxyapigenin. In some embodiments, the plant extract includes Compound 30.

In some embodiments, the oral composition includes an extract from one or more plant source. In some embodiments, the oral composition includes an extract from two or more plant sources. In some embodiments, the oral composition includes an extract from three or more plant sources.

In some embodiments, the extract as described herein includes juices, infusions, and fermentation residues. The products fermentation plant-derived extracts or processed fractions thereof find use in consumable compositions such as health-promoting compositions or tonics for human and animals.

In some embodiments, the composition includes one or more compounds or an extract of the present disclosure at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values. In some embodiments, the composition includes from about 0.1% to about 10% of one or more compounds or an extract as described herein. In some embodiments, the composition includes from about 0.1% to about 5% of one or more compounds or an extract as described herein. In some embodiments, the composition includes from about 1% to about 10% of one or more compounds or an extract as described herein. In some embodiments, when one or more compounds or an extract of the present disclosure is provided or administered as pharmaceuticals, nutraceuticals, or dietary supplements to humans and animals, they can be given per se or as a composition containing, for example, 0.1 to 99% active ingredient in combination with an acceptable carrier. In some embodiments, the compound or extract of the present disclosure may be provided or administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values. In some embodiments, the plant extracts described herein include one or more compounds selected from Table A. In some embodiments, the compositions described herein include one or more compounds selected from Table A. In some embodiments, the one or more compounds selected from Table A include Compounds 12, 13, 14, 15, 16, 17, 18, 19, 20, 21, 22, 23, 24, 25, 26, 27, 28, 29, 30, 31, 33, 34, 35, or a combination thereof.

In some embodiments, the composition may include one or more compounds as described herein and a suitable carrier or excipient. In some embodiments, the one or more compounds described herein may be in a ratio from about 10:1 to about 1:10, or ranges including and/or spanning the aforementioned values. In some embodiments, the one or more compounds described herein may be in a ratio from about 10:1, 9.5:1, 8.5:1, 8.0:1, 7.5:1, 7:1, 6.5:1, 6:1, 5.5:1, 5:1, 4.5:1, 4:1, 3.5:1, 3.0:1, 2.5:1, 2.0:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10. In some embodiments, the composition includes one or more compounds described in Table A. In some embodiments, the one or more compounds selected from Table A include Compounds 12, 13, 14, 15, 16, 17, 18, 19, 20, 21, 22, 23, 24, 25, 26, 27, 28, 29, 30, 31, 33, 34, 35, or a combination thereof.

In some embodiments, the disclosure provided herein provides for a composition comprising one or more compounds as described herein and a carrier. In some embodiments, the carrier is an acceptable carrier suitable for oral consumption. In some embodiments, the acceptable carrier is a pharmaceutically acceptable carrier. In some embodiments, the acceptable carrier is a food grade carrier. In some embodiments, the one or more compounds are substantially pure compounds. In some embodiments, the composition is an oral composition. The term “carrier” as used herein means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier should be compatible with the other ingredients of the formulation and not injurious to the subject. Some examples of materials that can serve as carriers include: (1) sugars, such as lactose, glucose, fructose, maltose, and sucrose; (2) starches, such as corn starch, tapioca starch, rice starch, wheat starch, and potato starch; (3) cellulose, and its derivatives, such as sodium carboxymethyl cellulose, microcrystalline cellulose, methylcellulose, ethyl cellulose, cellulose acetate, and hydroxyl propyl methyl cellulose; (4) powdered tragacanth; (5) malt; (6) gelatin; (7) talc; (8) excipients, such as cocoa butter, carnauba wax, beeswax, paraffin wax, candelilla wax, shellac wax, rice bran wax, and suppository waxes; (9) oils, such as peanut oil, cottonseed oil, safflower oil, sesame oil, olive oil, corn oil and soybean oil; (10) glycols, such as propylene glycol and glycerol (glycerin); (11) polyols, such as glycerin, xylitol, erythritol, maltitol, isomalt, lactitol, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) lipids and/or phospholipids; and (23) other nontoxic compatible substances employed in conventional formulations. Examples of food grade carriers include vegetable proteins, soy proteins, ion exchangers, soft gels, oils, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, PEG, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and mixtures thereof. Other examples of pharmaceutically acceptable carriers may be found, for example, in AN'EL'S PHARMACEUTICAL DOSAGE FORMS AND DRUG DELIVERY SYSTEMS (9th Ed., Lippincott Williams and Wilkins (2010)), PHARMACEUTICAL SCIENCES (8th Ed., Mack Publishing Co. (1990) or REMINGTON: THE SCIENCE AND PRACTICE OF PHARMACY (1st Ed., Lippincott Williams & Wilkins (2005)), which are incorporated in their entirety. In some embodiments, the carrier is about 0.5% to about 80% (w/w) of the composition. In some embodiments, the carrier is about 0.5%, about 1.0%, about 1.5%, about 2.0%, about 2.5%, about 3.0%, about 3.5%, about 4.0%, about 4.5%, about 5.0%, about 7.5%, about 10%, about 15%, about 20%, about 25%, about 30%, about 35%, about 40%, about 45%, about 50%, about 55%, about 60%, about 70%, about 75%, about 80% (w/w) of the composition, or ranges including, between, and/or spanning the aforementioned values.

In some embodiments, the oral composition may further include a preservative. In some embodiments, the preservative may be in an amount from about 0.01%, 0.05%, 0.1%, 0.15%, 0.20%, 0.25%, 0.50%, 0.75%, 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0% w/w, or ranges including and/or spanning the aforementioned values. In some embodiments, the preservative may be, but not limited to, the group selected from sodium benzoate, methyl parabens, propyl parabens, sodium nitrite, sulfur dioxide, sodium sorbate, ascorbic acid, tocopherols, calcium propionate, citric acid, sorbic acid, butylated hydroxtoluene (BHT), butylated hydroxyanisole (BHA), alcohol, such as ethyl alcohol, rosemary extract, and potassium sorbate. Other suitable preservatives include, but are not limited to, salts of edetate, (also known as salts of ethylenediaminetetraacetic acid, or EDTA, such a disodium EDTA).

In some embodiments, the oral composition may further include a sweetener. In some embodiments, the sweetener may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the sweetener may include a natural sweetener. In some embodiments, the sweetener may include a synthetic sweetener. Non-limiting examples of sweeteners include sucrose, dextrose, maltose, dextrin, dried invert sugar, fructose, high fructose corn syrup, levulose, galactose, corn syrup solids, tagatose, polyols (e.g., sorbitol, mannitol, xylitol, lactitol, erythritol, and maltitol), hydrogenated starch hydrolysates, isomalt, trehalose, and mixtures thereof.

In some embodiments, the oral composition may further include a flavoring agent. In some embodiments, the flavoring agent may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the flavoring agent may include natural or artificial flavors. In a particular embodiment, the flavoring agent comprises an essential oil, such as an oil derived from a plant or a fruit, peppermint oil, spearmint oil, other mint oils, clove oil, cinnamon oil, oil of wintergreen, bay, thyme, cedar leaf, nutmeg, allspice, sage, mace, and almonds. In another particular embodiment, the flavoring agent comprises a plant extract or a fruit essence such as apple, banana, watermelon, pear, peach, grape, strawberry, raspberry, cherry, plum, pineapple, apricot, and mixtures thereof. In still another particular embodiment, the flavoring agent comprises a citrus flavor, such as an extract, essence, or oil of lemon, lime, orange, tangerine, grapefruit, citron, or kumquat.

In some embodiments, the oral composition may further include a coloring agent. In some embodiments, the coloring agent may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values.

In some embodiments, the oral composition may further include at least one additive. In some embodiments, the additive may be in an amount from about 1.0%, 1.25%, 1.50%, 1.75%, 2.0%, 2.25%, 2.50%, 2.75%, 3.0%, 3.25%, 3.5%, 3.75%, 4.0%, 4.25%, 4.5%, 4.75%, 5.0%, 6.0%, 7.0%, 8.0%, 9.0%, 10.0% w/w, or ranges including and/or spanning the aforementioned values. In some embodiments, the at least one additive may be an emulsifier. In some embodiments, the additive may include one or more vitamin. In some embodiments, the additive may include one or more minerals. In some embodiments, the additive may include a stabilizer.

In some embodiments, the composition as described herein may include two or more compounds of Formula (I), (IA), (IB), (II), or (III). In some embodiments, the two or more compounds described herein may be in a ratio from about 10:1 to about 1:10, or ranges including and/or spanning the aforementioned values. In some embodiments, the two or more compounds described herein may be in a ratio from about 10:1, 9.5:1, 8.5:1, 8.0:1, 7.5:1, 7:1, 6.5:1, 6:1, 5.5:1, 5:1, 4.5:1, 4:1, 3.5:1, 3.0:1, 2.5:1, 2.0:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10. In some embodiments, the two or more compounds may be selected from Table A, a salt, or isomer thereof. In some embodiments, the two or more compounds selected from Table A include Compounds 12, 13, 14, 15, 16, 17, 18, 19, 20, 21, 22, 23, 24, 25, 26, 27, 28, 29, 30, 31, 33, 34, 35, or a combination thereof. In some embodiments, one of the two or more compounds includes Compound 12. In some embodiments, one of the two or more compounds includes Compound 13. In some embodiments, one of the two or more compounds includes Compound 14. In some embodiments, one of the two or more compounds includes Compound 15. In some embodiments, one of the two or more compounds includes Compound 16. In some embodiments, one of the two or more compounds includes Compound 17. In some embodiments, one of the two or more compounds includes Compound 18. In some embodiments, one of the two or more compounds includes Compound 19. In some embodiments, one of the two or more compounds includes Compound 20. In some embodiments, one of the two or more compounds includes Compound 21. In some embodiments, one of the two or more compounds includes Compound 22. In some embodiments, one of the two or more compounds includes Compound 23. In some embodiments, one of the two or more compounds includes Compound 24. In some embodiments, one of the two or more compounds includes Compound 25. In some embodiments, one of the two or more compounds includes Compound 26. In some embodiments, one of the two or more compounds includes Compound 27. In some embodiments, one of the two or more compounds includes Compound 28. In some embodiments, one of the two or more compounds includes Compound 29. In some embodiments, one of the two or more compounds includes Compound 30. In some embodiments, one of the two or more compounds includes Compound 31. In some embodiments, one of the two or more compounds includes Compound 32. In some embodiments, one of the two or more compounds includes Compound 33. In some embodiments, one of the two or more compounds includes Compound 34. In some embodiments, one of the two or more compounds includes Compound 35. In some embodiments, one of the two or more compounds includes Compound 36. In some embodiments, one of the two or more compounds includes Compound 37. In some embodiments, one of the two or more compounds includes Compound 38. In some embodiments, the two or more compounds include at least two of Compounds 14, 22, 23, 26, and 30. In some embodiments, the two or more compounds are in a ratio from about 10:1 to about 1:10. In some embodiments, the two or more compounds are in a ratio from about 5:1 to about 1:5. In some embodiments, the two or more compounds are in a ratio from about 2.5:1 to about 2:1. In some embodiments, the two or more compounds are in a ratio from about 2.5:1 to about 1:2.5. In some embodiments, the two or more compounds are in a ratio from about 5:1. In some embodiments, the two or more compounds are in a ratio from about 4:1. In some embodiments, the two or more compounds are in a ratio from about 3:1. In some embodiments, the two or more compounds are in a ratio from about 2:1. In some embodiments, the two or more compounds are in a ratio from about 1:1. In some embodiments, the two or more compounds are in a ratio from about 1:2. In some embodiments, the two or more compounds are in a ratio from about 1:3. In some embodiments, the two or more compounds are in a ratio from about 1:4. In some embodiments, the two or more compounds are in a ratio from about 1:5. In some embodiments, the oral composition includes a compound of Formula (III) but does not include a compound selected from the group of Compounds 30, 31, or a combination thereof. In some embodiments, the oral composition includes two or more compounds of Formula (I), (IA), (IB), (II), or (III) but does not include a compound selected from the group of Compounds 12, 13, 14, 15, 16, 17, 18, 19, 20, 21, 22, 23, 24, 25, 26, 27, 28, 29, 30, 31, 33, 34, 35, or a combination thereof. In some embodiments, the oral composition consists essentially of Compounds 12, 13, 14, 15, 16, 17, 18, 19, 20, 21, 22, 23, 24, 25, 26, 27, 28, 29, 30, 31, 33, 34, 35, or a combination thereof, unbound to a lignocellulosic material. In some embodiments, the oral composition includes two or more compounds selected from Compounds 12, 13, 14, 15, 16, 17, 18, 19, 20, 21, 22, 23, 24, 25, 26, 27, 28, 29, 30, 31, 33, 34, 35, or a combination thereof, with the proviso that the two or more compound is not bound to lignin, cellulose, and/or pectin in the oral composition. In some embodiments, the oral composition includes two or more compounds selected from Compounds 12, 13, 14, 15, 16, 17, 18, 19, 20, 21, 22, 23, 24, 25, 26, 27, 28, 29, 30, 31, 33, 34, 35, or a combination thereof, with the proviso that the two or more compound is not from a naturally source extract or extracted from a natural source, for example, a plant.

In some embodiments, the oral composition may consist essentially of one more compounds as described herein unbound to a lignocellulosic material. Lignocellulosic material refers to a composite material composed primarily of lignin, cellulose, and hemicellulose. These are naturally occurring polymers found in the cell walls of plants. Lignocellulosic materials are characterized by their rigid structure, high strength, and resistance to decomposition. This material is commonly derived from wood, grasses, and the inedible parts of plants. In some embodiments, the composition does not include lignin, cellulose, and/or pectin. In some embodiments, the composition does not include an extract from a natural source.

In some embodiments, the oral composition includes a compound of Formula (I) selected from the group of Compounds 12, 13, 14, 15, 20, 21, 22, 23, 24, 25, 26, 27, 28, 29, 33, 34, 35. In some embodiments, the oral composition includes a compound of Formula (I) selected from the group of Compounds 14, 22, 23, and 26. In some embodiments, the oral composition includes a compound of Formula (I) selected from the group of Compounds 22 and 23. In some embodiments, the oral composition includes a compound of Formula (I) and a compound of Formula (II). In some embodiments, the compound of Formula (II) is selected from the group of Compounds 16, 17, 18, and 19. In some embodiments, the oral composition includes a compound of Formula (I) and a compound of Formula (III). In some embodiments, the compound of Formula (III) is Compound 30 or 31. In some embodiments, the oral composition includes a compound of Formula (II) and a compound of Formula (III). In some embodiments, the oral composition includes a two or more compounds selected from Table A. In some embodiments, the oral composition includes a compound of Formula (I) but does not include a compound selected from the group of Compounds 12, 13, 14, 15, 20, 21, 22, 23, 24, 25, 26, 27, 28, 29, 33, 34, 35, or a combination thereof. In some embodiments, the oral composition includes a compound of Formula (II) but does not include a compound selected from the group of Compounds 16, 17, 18, 19, or a combination thereof. In some embodiments, the oral composition includes a compound of Formula (III) but does not include a compound selected from the group of Compounds 30, 31, or a combination thereof. In some embodiments, the oral composition includes one or more compounds of Formula (I), (IA), (IB), (II), or (III) but does not include a compound selected from the group of Compounds 12, 13, 14, 15, 16, 17, 18, 19, 20, 21, 22, 23, 24, 25, 26, 27, 28, 29, 30, 31, 33, 34, 35, or a combination thereof. In some embodiments, the oral composition consists essentially of Compounds 12, 13, 14, 15, 16, 17, 18, 19, 20, 21, 22, 23, 24, 25, 26, 27, 28, 29, 30, 31, 33, 34, 35, or a combination thereof, unbound to a lignocellulosic material. In some embodiments, the oral composition includes one or more compounds selected from Compounds 12, 13, 14, 15, 16, 17, 18, 19, 20, 21, 22, 23, 24, 25, 26, 27, 28, 29, 30, 31, 33, 34, 35, or a combination thereof, with the proviso that the one or more compound is not bound to lignin, cellulose, and/or pectin in the oral composition. In some embodiments, the oral composition includes one or more compounds selected from Compounds 12, 13, 14, 15, 16, 17, 18, 19, 20, 21, 22, 23, 24, 25, 26, 27, 28, 29, 30, 31, 33, 34, 35, or a combination thereof, with the proviso that the one or more compound is not from a naturally source extract or extracted from a natural source, for example, a plant.

### Formulations

Aspects of the disclosure relate to formulation including one or more compounds as described herein. In some embodiments, a substantially pure compound or extract comprising one or more compounds of this disclosure can be combined with a carrier and provided in any suitable form for consumption by, provided to, or administration to a subject. In some embodiments, the one or more compounds or extract comprising one or more compounds is added as an exogenous ingredient or additive to a consumable. Suitable consumable forms include, but are not limited to, a dietary supplement, food ingredient or additive, a food product, a medical food, nutraceutical or pharmaceutical composition. In some embodiments, the one or more compounds or extract comprising one or more compounds is provided in either a liquid, granular, or powder form. In some embodiments, the formulation may be a food product.

A composition comprising a plant extract or a compound of this disclosure can be combined with a carrier and provided in any suitable form for consumption by or administration to a subject. In this respect, the plant extract or compound is added as an exogenous ingredient or additive to the consumable. Suitable consumable forms include, but are not limited to, a dietary supplement, food ingredient or additive, a medical food, nutraceutical or pharmaceutical composition. In some embodiments, the plant extract or compound is provided in either a liquid or powder form.

A food ingredient or additive is an edible substance intended to result, directly or indirectly, in becoming a component of or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound. In some embodiments, the food product includes one or more compounds as described herein and a fiber source. In some embodiments, the food product includes one or more compounds as described herein. In some embodiments, the food product is a food bar. In some embodiments, the food bar comprises from about 0.01% to about 20% (w/w) of one or more compounds as described herein. In some embodiments, the food bar comprises from about 0.1% to about 10% (w/w) of one or more compounds as described herein. In some embodiments, the food product is a crisp. In some embodiments, the food crisp comprises from about 0.01% to about 20% (w/w) of one or more compounds as described herein. In some embodiments, the food crisp comprises from about 0.1% to about 10% (w/w) of one or more compounds as described herein.

The compounds, compositions and extracts of the present disclosure can be prepared as nutritional formulations such as foods, including medical or functional foods. A “medical or functional food” is defined as being consumed as part of a usual diet but which has been demonstrated to have physiological benefits and/or to reduce the risk of a disease or condition such as a chronic disease, beyond basic nutritional functions. By way of example, but not limitation, medical foods and functional foods may include one or more of the following ingredients: a compound described herein, a composition described herein, an extract described herein, vitamins, minerals, herbs, botanicals, amino acids, dietary substances intended to supplement the diet by increasing total dietary intake, concentrates, metabolites, constituents, extracts or combinations of any of the foregoing. Dietary supplements may also be incorporated into food stuffs, such as functional foods designed to promote health or to prevent disease or disorders. If administered as a medicinal preparation, the composition can be administered, either as a prophylaxis or treatment, to a patient in any of a number of methods. In some embodiments, the compositions described herein may be administered alone or in combination with other additives or pharmaceutical agents and can be combined with a physiologically acceptable carrier thereof. The effective amount and method of administration and aim of the particular formulation can vary based on the individual subject, the stage of the disease or condition, and other factors evident to one skilled in the art. In the case of a pharmaceutical formulation as well as a nutraceutical formulation, during the course of the treatment, the concentration of the subject compositions may be monitored (for example, blood plasma levels may be monitored) to ensure that the desired level is maintained.

In some embodiments, food product includes a carbohydrate. A variety of carbohydrates are used in food products, such as various sugars and starches. Carbohydrates are an important source of energy for the body, including complex carbohydrates (like whole grains and vegetables) and simple carbohydrates (like sugar and refined grains). There are several common carbohydrates that are used in food products, including: starch, sugar, fructose, maltodextrin, dextrose, corn syrup, oligosaccharides, cellulose, complex carbohydrates, such as inulin that may be added as a prebiotic fiber in processed foods. According to the disclosure, the concentration of carbohydrates may vary depending on the intended use of a product. In some embodiments, carbohydrates may comprise from about 1% by weight to about 95% by weight of a food product. In some embodiments, carbohydrates may comprise from about 1% by weight to about 95% by weight of a food product. In some embodiments, a food product may comprise carbohydrates in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a sugar. Sugars, such as high fructose corn syrup, sucrose, glucose syrup, dextrose, and other sweeteners are often added to processed foods to make them more palatable. Additionally, sugar alcohols or maltodextrins, may be added to food products to reduce caloric content and/or to enhance nutritional profile of the product. In some embodiments, a food product may comprise one or a plurality of saccharides that are slowly or incompletely digested by humans, if not totally indigestible. These sugars can include isomaltose, panose and branched oligomers having a degree of polymerization of four or greater. Additional non-limiting examples of sugars include sucrose, HFCS, fructose, brown sugar (which can be either partially or fully refined), powdered sugar (also known as confectioner's sugar), high fructose corn syrup, honey, molasses, maple syrup, agave nectar, coconut sugar, date sugar, fruit juice concentrates, maltodextrin, dextrose, glucose syrup, maple syrup, molasses, and lactose. According to the disclosure, the concentration of sugars may vary depending on the intended use of a product. In some embodiments, sugar may comprise from about 1% by weight to about 95% by weight of a food product. In some embodiments, the sugar(s) component comprises from about 2% to about 10% by weight of the composition. In some embodiments, sugars may comprise from about 1% by weight to about 95% by weight of a food product. In some embodiments, a food product may comprise sugars in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product includes a fiber source. In some embodiments, the food product includes from about 5% to about 30% (w/w) fiber. In some embodiments, the food product includes at least 10% fiber. In some embodiments, the food product includes at least 15% fiber. In some embodiments, the food product includes at least 20% fiber. In some embodiments, the food product includes at least 30% fiber. In some embodiments, the fiber may be selected from psyllium husk, inulin, beta-glucans, acacia fiber, pectin, wheat dextrin, cellulose, fructooligosaccharides, guar gum, fruit fiber, or a combination thereof. In some embodiments, the fiber is a soluble dietary fiber.

In some embodiments, the food product may include a protein. Proteins may be included in a range of concentrations depending on the product. Protein sources can include soy protein, soy flour, soy protein isolate, whey protein isolate, casein, gelatin, legume protein isolates, soy protein concentrate, egg albumin or egg white, wheat protein concentrate, legume protein concentrates and mixtures thereof. In some embodiments, meat products like beef jerky, sausages, or meatballs, may include proteins as a primary ingredient. Proteins may be included at concentrations ranging from about 10% to about 25% of the total ingredients. In dairy products like cheese, yogurt, or milk, proteins like casein or whey may be the primary ingredient. Proteins may be included at concentrations ranging from about 3% to about 15% of the total ingredients. In plant-based products like tofu, tempeh, or seitan, proteins from soy, peas, legumes, or wheat may be primary ingredient. Proteins may be included at concentrations ranging from about 10% to about 25% of the total ingredients. In energy bars and protein bars, proteins like whey or soy, or any protein according to the disclosure may be added to provide a source of protein. Proteins may be included at concentrations ranging from about 10% to about 25% of the total ingredients. According to the disclosure, the concentration of proteins may vary depending on the intended use of a product. In some embodiments, proteins may comprise from about 1% by weight to about 95% by weight of a food product. In some embodiments, proteins may comprise from about 1% by weight to about 95% by weight of a food product. In some embodiments, a food product may comprise proteins in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a starch. In some embodiments, food products may additionally comprise a starch ingredient(s) in amounts sufficient to provide about 5% to 45%, or about 10%-30%, or about 15%-25 starch in the food products. Starchy components may include not only pure added cereal flours or other granulations but also any starchy fraction provided by other ingredients such as oat bran or soy protein. In some embodiments, a starch may comprise any conventionally employed starch or cereal flour ingredient, for use in a ready-to-eat cereal. Exemplary suitable starchy cereals include cereal flours from major cereal grains including wheat, rice, corn (maize), oats, barley, rye, or starch fractions isolated from the cereal flowers including, for example cornstarch, wheat starch, rice starch, and various treated starches including pre-gelatinized starches and/or modified starches.

In some embodiments, the food product may include a flour. Several types of flour may be used according to the disclosure. For example, common types of flour used in food products, include all-purpose flour (which like most other flour may include protein content), Whole wheat flour, Bread flour, Cake flour, pastry flour, self-rising flour, and gluten-free flour. According to the disclosure, the concentration of flour may vary depending on the intended use of a product. For example, in baked goods like bread, cakes, and cookies, flour is typically a main ingredient and may be included at concentrations ranging from 50% to 100% of the total dry ingredients. In soups and sauces, flour is often used as a thickener and may be included at concentrations ranging from 1% to 5% of the total ingredients. Batter and breading: In fried foods like chicken or fish, flour is often used as part of the batter or breading and may be included at concentrations ranging from 20% to 50% of the total dry ingredients. In snack foods, like crackers and chips, flour may be included at concentrations ranging from 30% to 70% of the total ingredients. In some embodiments, flour may comprise from about 1% by weight to about 95% by weight of a food product. In some embodiments, flours may comprise from about 1% by weight to about 95% by weight of a food product. In some embodiments, a food product may comprise flour in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include a fat. In some embodiments, the food product may include an oil. In some embodiments, the food product may include one or more fats or oils. Several types of fats may be used according to the disclosure. For example, common types of fats include butter, margarine, vegetable oils, shortening, and lard. According to the disclosure, the concentration of fats may vary depending on the intended use of a product. In baked goods like cakes, cookies, and pastries, fats like butter, shortening, or oil are often used to provide moisture, flavor, and texture. In some embodiments, Fats may be included at concentrations ranging from 10% to 30% of the total ingredients. In some embodiments, like in sauces and dressings, fats like olive oil or mayonnaise are often used to provide flavor and texture. In some embodiments, Fats may be included at concentrations ranging from 10% to 30% of the total ingredients. In fried foods like chicken or French fries, fats like vegetable oil or lard are used for frying and may be included at concentrations ranging from 30% to 60% of the total ingredients. In snack foods like chips and crackers, fats like vegetable oil or palm oil are often used to provide flavor and texture. In some embodiments, Fats may be included at concentrations ranging from 10% to 30% of the total ingredients. In some embodiments, fats may comprise from about 1% by weight to about 95% by weight of a food product. In some embodiments, fats may comprise from about 1% by weight to about 95% by weight of a food product. In some embodiments, a food product may comprise fat in a range with high and low values independently selected from about 1%, 3%, 5%, 8%, 10%, 15%, 20%, 25%, 30%, 35%, 40%, 45%, 50%, 55%, 60%, 65%, 70%, 75%, 80%, 85%, 90%, 95% or ranges including, between, and/or spanning the aforementioned values.

In some embodiments, the food product may include one or more additional ingredients. If desired, the disclosed food products may additionally include a variety of materials designed to improve their aesthetic or nutritional qualities. These adjuvant materials can include vitamin and/or mineral fortification, colors, flavors, sweetener(s), and mixtures thereof. The precise ingredient concentration may vary. Generally, however, such materials can each include about 0.01% to about 5%, or about 0.1% to 2% dry weight of a food product. One especially useful material is common salt. In some embodiments, salt comprises about 0.1 to 5%, or about 0.5 to 4.0% of the food products.

In some embodiments, the food product is derived from a plant. In some embodiments, the food product is derived from hemp hulls. In some embodiments, the food product is a solid food. In some embodiments, the food product is a semi-solid food. In some embodiments, the food product is a puffed product, a bakery product, a pressed cake, a cooked product, a food bar, a cereal, a crisp, or a spread.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use. In some embodiments, the composition as described herein is provided as a dietary supplement. In some embodiments, the dietary supplement is provided as a capsule, tablet, powder, or in a liquid form. In some embodiments, the composition as described herein is provided as a nutritional supplement. In some embodiments, the nutritional supplement is provided as a capsule, tablet, powder, or in a liquid form. In some embodiments, the composition as described herein is provided as a pharmaceutical. In some embodiments, the pharmaceutical is provided as a capsule, tablet, powder, or in a liquid form.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound as described herein, or a pharmaceutically acceptable salt thereof, can be administered orally. In some embodiments, a compound described herein, including a compound of Formula (I), (IA), (IB), (II), (III), or a salt thereof, can be administered orally. In some embodiments, a compound described herein, including a compound of Formula (I), (IA), (IB), (II), (III), or a salt thereof, can be provided in a form for oral consumption.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The oral compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example, syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In some embodiments, an oral composition as described herein is administered at a dose in the range of about 0.1-200 mg/kg body weight. In some embodiments, an oral composition as described herein is administered at a dose in the range of about 0.1-1, 0.5-1, 0.1-10, 0.5-10, 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500,60-600, 60-700,60-800, 60-900,60-1000, 70-80,70-90, 70-100,70-200, 70-300,70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, an oral composition as described herein is administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 80, 90, or 95 mg/kg of the body weight. In some embodiments, an oral composition as described herein is administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, an oral composition as described herein is administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of a subjects body weight.

In some embodiments, an oral composition as described herein dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg-50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, an oral composition as described herein is administered at about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, an oral composition as described herein dose is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg. about 400 mg, about 500 mg, about 600 mg, about 700 mg, about 800 mg, about 900 mg, or about 1000 mg. In some embodiments, an oral composition as described herein dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

The liquid forms in which the compound or extract of the disclosure is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

When a compound or extract of the present disclosure is administered as pharmaceuticals, nutraceuticals, or dietary supplements to humans and animals, they can be given per se or as a composition containing, for example, 0.1 to 99% active ingredient in combination with an acceptable carrier. In some embodiments, the one or more compounds or extract of the present disclosure may be administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values.

In some embodiments, the fortified food, food supplement, or medical food may further include one or more of a protein, carbohydrate and/or fat that is used in the body, preferably a human body, to sustain growth, repair, and vital processes, and to furnish energy. When provided as a fortified food, food supplement, or medical food, the compositions comprise selected compounds, compositions, or extracts of the disclosure such that they can be eaten at or about the same time as a food. In some embodiments, the fortified food, food supplement, or medical food are generally eaten within about one hour before or after the food is eaten. In some embodiments, the fortified food, food supplement, or medical food are generally eaten within about fifteen minutes before or after the food is eaten. In some embodiments, the fortified food, food supplement, or medical food are generally eaten within about five minutes before or after the food is eaten. In some embodiments, dietary supplement, or nutritional supplement also be eaten at the same time as the food, or even with fortified food, a food supplement, or medical food.

### Methods and Uses

In aspects, this disclosure provides methods and uses for improving, restoring, modulating, or maintaining sleep. In accordance with such methods, an effective amount of one or more compounds or a composition of this disclosure is provided to a subject in need thereof so that the subject's sleep is improved, restored, modulated, or maintained. The term “subject” as used herein refers to an animal, preferably a mammal. In some embodiments, the subject is a veterinary, companion, farm, laboratory or zoological animal. In other embodiments, the subject is a human.

Several signaling molecules that play a role in sleep have been identified. Various neurotransmitters, including noradrenaline, acetylcholine, histamine, dopamine, serotonin, and the neuropeptides orexin A and B (also referred to as hypocretins), promote wakefulness. Orexins are neuropeptides involved in the regulation of the sleep-wake cycle, feeding pattern, energy balance, and stress in mammals. Dysregulation or loss of orexin signaling has been linked to narcolepsy. In some embodiments, the plant extract or compound as described herein are orexin receptor antagonists. In some embodiments, the plant extract or compound as described herein include an orexin receptor 2 (OX2) antagonist. In some embodiments, the plant extract or compound as described herein include an orexin receptor 1 (OX1) antagonist. In some embodiments, the plant extract or compound as described herein are capable of being an antagonist for both orexin receptor 1 and orexin receptor 2. In some embodiments, the compounds or compositions described herein may be useful in a method of antagonizing orexin receptor activity in a subject in need of such inhibition comprising the administration of an effective amount of the compound. The utility of the compounds in accordance with the present disclosure as OX1 and/or OX2 antagonists may be readily determined without undue experimentation by methodology known in the art, including the “FLIPR Ca²⁺ Flux Assay” (Okumura et al., Biochem. Biophys. Res. Comm. 280:976-981, 2001).

Calcium influx may be important for generating ON/OFF state oscillations and REM sleep. In some embodiments, the plant extract or compound as described herein provides a decrease in calcium influx in a subject. In some embodiments, a method for decreasing calcium influx in a subject includes administering a composition to the subject as described herein.

Sleep deprivation leads to ER stress and endoplasmic reticulum kinase (pERK) pathway activation. In some embodiments, the plant extract or compound as described herein provides a decrease in pERK 1/2 phosphorylation. In some embodiments, a method for deactivating a subject's pERK 1/2 phosphorylation includes administering a composition as provided herein.

In some embodiments, a method includes speedily inducing sleep in a subject by administering composition as described herein. In some embodiments, the subject falls asleep more quickly from about 1.0%, 2.0%, 3.0%, 4.0%, 5.0%, 6.0%, 7.0%, 8.0%, 9.0%, 10.0%, 15.0%, 20%, 25, 0%, 50.0%, 75.0%, or ranges including and/or spanning the aforementioned values.

In some embodiments, a method includes promoting good quality sleep in a subject by administering a composition as described herein. In some embodiments, the subject quality of sleep improves from about 1.0%, 2.0%, 3.0%, 4.0%, 5.0%, 6.0%, 7.0%, 8.0%, 9.0%, 10.0%, 15.0%, 20%, 25, 0%, 50.0%, 75.0%, or ranges including and/or spanning the aforementioned values.

In some embodiments, a method includes providing restful sleep in a subject by administering a composition as described herein. In some embodiments, the subjects restful sleep improves from about 1.0%, 2.0%, 3.0%, 4.0%, 5.0%, 6.0%, 7.0%, 8.0%, 9.0%, 10.0%, 15.0%, 20%, 25, 0%, 50.0%, 75.0%, or ranges including and/or spanning the aforementioned values.

In some embodiments, a method includes providing a full night's sleep in a subject by administering a composition as described herein. In some embodiments, the subject sleeps longer on average by an additional about 15 min, 30 min, 45 min, 60 min, 75 min, 90 min, 105 min, 120 min, 135 min, 150 min, 165 min, 180 min, or ranges including and/or spanning the aforementioned values. In some embodiments, a method includes decreasing the time to sleep onset in a subject by administering a composition as described herein. In some embodiments, the subject decreases the time to sleep onset on average from about 15 min, 30 min, 45 min, 60 min, 75 min, 90 min, 105 min, 120 min, 135 min, 150 min, 165 min, 180 min, or ranges including and/or spanning the aforementioned values.

In some embodiments, a method includes increasing Slow-Wave-Sleep 1-4 in a subject by administering a composition as described herein. In some embodiments, a method includes increasing REM sleep in a subject by administering a composition as described herein. In some embodiments, a method includes increasing deep sleep in a subject by administering a composition as described herein. There are many different periods of sleep a person goes through. These include Slow-Wave-Sleep 1 (SWS1), Slow-Wave-Sleep 2 (SWS 2), Slow-Wave-Sleep 3 (SWS 3) Slow-Wave-Sleep 4 (SWS 4) and Rapid Eye Movement (REM). SWS 1 and SWS 2 are both periods of light sleep where it is relatively easy to wake someone up. Light sleep is usually more frequent in the second half of sleep. SWS 3 and SWS 4 are both periods of deep sleep, where it is difficult to wake the sleeper. Deep sleep is more frequent in the first half of sleep and each period will get shorter each time afterward. REM is a period of sleep in which people have their most vivid dreams. The wave patterns are similar to the patterns in which a person is awake. For healthy people, SWS1 will occupy about 5% of sleep, SWS2 will occupy about 50% of sleep, SWS3 will occupy about 10% of sleep, SWS4 will occupy about 10% of sleep and REM will occupy about 25% of sleep. In some embodiments, the method may increase the Slow-Wave-Sleep 1 by about 1% to about 5% of the subject's sleep. In some embodiments, the method may increase the Slow-Wave-Sleep 2 by about 10% to about 50% of the subject's sleep. In some embodiments, the method may increase the Slow-Wave-Sleep 3 by about 2% to about 10% of the subject's sleep. In some embodiments, the method may increase the Slow-Wave-Sleep 4 by about 2% to about 10% of the subject's sleep. In some embodiments, the method may increase the REM by about 10% to about 25% of the subject's sleep.

In some embodiments, a method improves a sleep disorder in a subject by administering to the subject a composition as described herein. Sleep disorders are described in the International Classification of Sleep Disorders (ICDS). ICDS-3 was published in 2014 and characterizes sleep disorders as belonging to one of the following classes: (1) Insomnias; (2) Sleep Related Breathing Disorders; (3) Central Disorders of Hypersomnolence; (3) Circadian Rhythm Sleep-Wake Disorders; (4) Parasomnias; (5) Sleep Related Movement Disorders. Accordingly, the sleep disorders to be treated by the oral composition described herein may include any sleep disorders from the classes (1) Insomnias; (2) Sleep Related Breathing Disorders; (3) Central Disorders of Hypersomnolence; (3) Circadian Rhythm Sleep-Wake Disorders; (4) Parasomnias; (5) Sleep Related Movement Disorders. In particular, the oral compositions may be effective in the treatment of a sleep disorder selected from: insomnia, narcolepsy, hypersomnia, sleep apnoea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, a sleep disorder associated with another disease or condition, or any other sleep disorder.

By “effective amount” it is meant an amount sufficient that when administered to the patient an amount of the drug is provided to achieve an effect. In the case of a therapeutic method, this effect may be the treatment of the sleep disorder. Therefore, the “effective amount” may be a “therapeutically effective amount”. By “therapeutically effective amount” it is meant an amount sufficient that when administered to the subject an amount of drug is provided to treat the disease or a symptom of the disease.

As used herein, the terms “treating”, “treatment”, “treat” and the like mean affecting a subject, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease. For example, a reference to “treating” a sleep disorder therefore could encompasses: (a) assisting the subject to fall asleep; (b) assisting the subject remain asleep once sleep has been achieved; (c) relieving or ameliorating the effects of the sleep disorder, e.g. enhancing wakefulness during non-sleep periods; or (d) preventing the sleep disorder from occurring in a subject predisposed to, or at risk of, the sleep disorder, so that the sleep disorder does not develop or occur in the subject, or develops in a less severe form.

In some embodiments, the method to treat insomnia in a subject includes administering one or more compounds as described herein. In some embodiments, the method to treat insomnia in a subject includes administering an oral composition as described herein. Symptoms and severity of insomnia may be measured by the Insomnia Severity Index (ISI) questionnaire. Typically, the ISI is administered by a clinician, nurse or researcher or may be self-administered by the patient. The ISI assesses both night-time and daytime components of insomnia and is available in several languages. The ISI asks seven questions each to be scored on a scale of 0-4 relating to (1) the difficulty in falling asleep, (2) difficulty staying asleep, (3) problems waking too early, (4) satisfaction relating to current sleep patterns, (5) perception as to how noticeable the sleep problem may be to others, (6) degree of concern regarding the sleep problem, and (7) the extent to which the sleep problem interferes with daily functioning. The methods may provide an improvement in one or more of these seven aspects of insomnia. In some embodiments of the methods of treatment, the subject to be treated may have an initial ISI score of 7 or more, in some cases, the initial ISI score may be 10 or more. In some embodiments, a method of treating insomnia may provide a reduction of the ISI score of the patient relative to an initial ISI score. This reduction in ISI score may be by 1, 2, 3, 4, 5, 6 or more units on the ISI scale, and preferably results in the patient having an ISI score of 7 or less after treatment.

The ISI may be used alone to assess the severity of the subject's insomnia or it may be used together with one or more other questionnaires, such as quality of life enjoyment and satisfaction questionnaire (Q-les-Q), work and social adjustment scale (WSAS), depression anxiety stress scale (DASS) questionnaire, dysfunctional beliefs about sleep (DBAS) questionnaire, multidimensional fatigue inventory questionnaire, and any other recognized questionnaire known in the field.

Typically the patient is assessed by one or more questionnaires prior to receiving treatment and then at regular intervals (e.g., an interval of 1, 2, 3, 4, 5, 6, 7 or 8 weeks) during the course of treatment. The assessment during treatment may begin 2 weeks after commencement of treatment.

In some embodiments, the treatment may be maintained for up to 14 days, for example, the treatment may be maintained for 1, 2, 3, 4, 5, 6, 7, 8, 9, 10, 11, 12, 13 or 14 days. In some embodiments, the treatment is maintained for longer than 14 days, for example, for 3 weeks, 1, 2, 3, 4, 5, 6, 12, 18, 24, 36 months or longer.

Typically, the oral composition is administered once daily preferably a short time before the patient attempts to sleep. In some embodiments, the oral composition is administered within about 2 hours of the patient attempting to sleep, for example, within about 1.5 hours, within about 1 hour or within about 30 minutes prior to sleep.

In some embodiments, the insomnia may be assessed by measuring one or more objective measures of sleep. Objective measures of sleep may be measured by polysomnography (PSG) and/or actigraphy. The objective measures of sleep may include measuring: Sleep Onset Latency (SO); Wake After Sleep Onset (WASO); Total Sleep Time (TST); Sleep Efficiency (SE); REM versus NREM sleep patterns including slow-wave sleep patterns and percentage of time in all sleep stages; sleep apnea; periodic limb movements; and combinations thereof. In some embodiments, a method of treating insomnia may provide an improvement in one or more objective measures of sleep, for example, 2, 3, 4, 5, 6 or more of the objective measures of sleep.

In some embodiments, the compounds or compositions of the present disclosure may be administered in combination with other compounds to improve sleep quality. In some embodiments, the compounds or compositions of the present disclosure may be administered in combination with other compounds to treat or prevent sleep disorders and sleep disturbances. In some embodiments, the other compounds may include, but are not limited to, sedatives, hypnotics, anxiolytics, antipsychotics, antianxiety agents, antihistamines, benzodiazepines, barbiturates, cyclopyrrolones, GABA agonists, 5HT-2 antagonists including 5HT-2A antagonists and 5HT-2A/2C antagonists, histamine antagonists including histamine H3 antagonists, histamine H3 inverse agonists, imidazopyridines, minor tranquilizers, melatonin agonists and antagonists, melatonergic agents, prokineticin agonists and antagonists, pyrazolopyrimidines, T-type calcium channel antagonists, triazolopyridines, and the like, such as: adinazolam, allobarbital, alonimid, alprazolam, amitriptyline, amobarbital, amoxapine, armodafinil, APD-125, bentazepam, benzoctamine, brotizolam, bupropion, busprione, butabarbital, butalbital, capromorelin, capuride, carbocloral, chloral betaine, chloral hydrate, chlordiazepoxide, clomipramine, clonazepam, cloperidone, clorazepate, clorethate, clozapine, conazepam, cyprazepam, desipramine, dexclamol, diazepam, dichloralphenazone, divalproex, diphenhydramine, doxepin, EMD-281014, eplivanserin, estazolam, eszopiclone, ethchlorynol, etomidate, fenobam, flunitrazepam, flurazepam, fluvoxamine, fluoxetine, fosazepam, gaboxadol, glutethimide, halazepam, hydroxyzine, ibutamoren, imipramine, indiplon, lithium, lorazepam, lormetazepam, LY-156735, maprotiline, MDL-100907, mecloqualone, melatonin, mephobarbital, meprobamate, methaqualone, methyprylon, midaflur, midazolam, modafinil, nefazodone, NGD-2-73, nisobamate, nitrazepam, nortriptyline, ornortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, TAK-375, temazepam, thioridazine, tiagabine, tracazolate, tranylcypromaine, trazodone, triazolam, trepipam, tricetamide, triclofos, trifluoperazine, trimetozine, trimipramine, uldazepam, venlafaxine, zaleplon, zolazepam, zopiclone, zolpidem, and salts thereof, and combinations thereof, and the like, or the compound of the present invention may be administered in conjunction with the use of physical methods such as with light therapy or electrical stimulation.

This disclosure also provides methods and uses for treating, improving, restoring, modulating, or maintaining a subject's weight. In accordance with such methods, an effective amount of one or more compounds or a composition of this disclosure is provided to a subject in need thereof so that the subject's weight is treated, improved, restored, modulated, or maintained. In some embodiments, the one or more compounds, plant extract or composition is present in an amount sufficient to promote health and wellness.

In some embodiments, a method for weight management in a subject includes administering to the subject one or more compounds or a composition as described herein. In some embodiments, the subject reduces and/or maintains body mass. In some embodiments, the body mass is reduced and/or maintained from the subject's original body mass from about 1.0%, 2.0%, 3.0%, 4.0%, 5.0%, 6.0%, 7.0%, 8.0%, 9.0%, 10.0%, 15.0%, 20%, or ranges including and/or spanning the aforementioned values.

In some embodiments, a method for appetite suppression includes in a subject administering to the subject a composition as described herein. In some embodiments, the subject feels more full after administration of one or more compounds or a composition as described herein from about 1.0%, 2.0%, 3.0%, 4.0%, 5.0%, 6.0%, 7.0%, 8.0%, 9.0%, 10.0%, 15.0%, 20%, or ranges including and/or spanning the aforementioned values.

In some embodiments, a method for curbing appetite in a subject includes administering to the subject a composition as described herein. In some embodiments, the subject appetite is reduced after administration of one or more compounds or a composition as described herein from about 1.0%, 2.0%, 3.0%, 4.0%, 5.0%, 6.0%, 7.0%, 8.0%, 9.0%, 10.0%, 15.0%, 20%, or ranges including and/or spanning the aforementioned values.

In some embodiments, a method for inducing a feeling of satiety in a subject includes administering to the subject a composition as described herein. In some embodiments, the subject's induced satiation is increased after administration of one or more compounds or a composition as described herein from about 1.0%, 2.0%, 3.0%, 4.0%, 5.0%, 6.0%, 7.0%, 8.0%, 9.0%, 10.0%, 15.0%, 20%, or ranges including and/or spanning the aforementioned values. Satiety and/or satiation can be evaluated using consumer surveys (e.g., for humans) that can demonstrate a statistically significant measure of increased satiation and/or satiety.

Also provided are methods for promoting weight loss by administering a composition as provided herein to a subject. In some embodiments, the subject reduces body mass after administration of one or more compounds or a composition as described herein from about 1.0%, 2.0%, 3.0%, 4.0%, 5.0%, 6.0%, 7.0%, 8.0%, 9.0%, 10.0%, 15.0%, 20%, or ranges including and/or spanning the aforementioned values. The amount and duration of such administration will depend on the individual's weight loss needs and health status, and can be evaluated by those having ordinary skill in the art. The subject's weight loss can be measured over time to determine if weight loss is occurring. Weight loss can be compared to a control animal not administered the ingestible composition.

### EXAMPLES

The following non-limiting examples are provided to further illustrate the present disclosure.

### Example 1

Dionex™ accelerated solvent extractor 350 was used for the extraction of plant material. Four 100 mL stainless steel ASE extraction cells were filled with 25 g of grinded plant tissues. The pre-set temperature was 60° C., the static extraction was run for a total of twelve extraction cycles. The solvents used for the extraction were 95% ethanol, ethyl acetate, and hexane. The solvent was removed, and the 95% ethanol extract was subjected to a bioassay-guided fractionation protocol. Briefly, the extract was dry-loaded onto C18 spherical flash column chromatography. Elution was conducted with a mobile phase consisting of water+0.1% formic acid (A) and CH₃CN+0.1% formic acid (B), following the gradient, 1% to 3% B in 3 min, increasing to 100% B in 22 min then maintaining 100% B for 7 min at a flow rate of 30 mL/min. The highly active fractions were pooled, and the polled sample was further purified by bioassay-guided fractionation using Kinetex reverse-phase C18 preparative column yielding costunulide.

### Example 2

This is a prophetic example. In this example, the extraction method of Example 1 to the plant tissues from Table 1.

TABLE 1

| Genus species | Plant Tissue(s) |
|---|---|
| Acorus gramineus | Leaf, pulp, fruit, bulb, seed, tuber, root, flower |
| Alloteropsis semialata | Leaf, pulp, fruit, bulb, seed, tuber, root, flower |
| Andrographis paniculata | Leaf, pulp, fruit, bulb, seed, tuber, root, flower |
| Arabidopsis thaliana | Leaf, pulp, fruit, bulb, seed, tuber, root, flower |
| Arachis hypogaea var. vulgaris | Leaf, pulp, fruit, bulb, seed, tuber, root, flower |
| Arnica montana, | Leaf, pulp, fruit, bulb, seed, tuber, root, flower |
| Artemisia annua | Leaf, pulp, fruit, bulb, seed, tuber, root, flower |
| Artemisia maritima | Leaf, pulp, fruit, bulb, seed, tuber, root, flower |
| Barnadesia spinosa | Leaf, pulp, fruit, bulb, seed, tuber, root, flower |
| Bletilla striata | Leaf, pulp, fruit, bulb, seed, tuber, root, flower |
| Brachypodium distachyon | Leaf, pulp, fruit, bulb, seed, tuber, root, flower |
| Camellia sinensis | Leaf, pulp, fruit, bulb, seed, tuber, root, flower |
| Cichorium intybus | Leaf, pulp, fruit, bulb, seed, tuber, root, flower |
| Clausena excavata | Leaf, pulp, fruit, bulb, seed, tuber, root, flower |
| Dolomiaea souliei | Leaf, pulp, fruit, bulb, seed, tuber, root, flower |
| Eupatorium perfoliatum | Leaf, pulp, fruit, bulb, seed, tuber, root, flower |
| Eruca vesicaria | Leaf, pulp, fruit, bulb, seed, tuber, root, flower |
| Fallopia multiflora | Leaf, pulp, fruit, bulb, seed, tuber, root, flower |
| Glebionis coronaria | Leaf, pulp, fruit, bulb, seed, tuber, root, flower |
| Glycine max | Leaf, pulp, fruit, bulb, seed, tuber, root, flower |
| Gossypium hirsutum | Leaf, pulp, fruit, bulb, seed, tuber, root, flower |
| Grindelia integrifolia | Leaf, pulp, fruit, bulb, seed, tuber, root, flower |
| Gynura procumbens | Leaf, pulp, fruit, bulb, seed, tuber, root, flower |
| Helianthus annuus | Leaf, pulp, fruit, bulb, seed, tuber, root, flower |
| Hordeum spontaneum | Leaf, pulp, fruit, bulb, seed, tuber, root, flower |
| Hordeum vulgare cv. Seco | Leaf, pulp, fruit, bulb, seed, tuber, root, flower |
| Hordeum vulgare subsp. vulgare | Leaf, pulp, fruit, bulb, seed, tuber, root, flower |
| Hypericum perforatum | Leaf, pulp, fruit, bulb, seed, tuber, root, flower |
| Inula helenium | Leaf, pulp, fruit, bulb, seed, tuber, root, flower |
| Lactuca sativa | Leaf, pulp, fruit, bulb, seed, tuber, root, flower |
| Laurus nobilis | Leaf, pulp, fruit, bulb, seed, tuber, root, flower |
| Lobelia chinensis | Leaf, pulp, fruit, bulb, seed, tuber, root, flower |
| Momordica cochinchinensis | Leaf, pulp, fruit, bulb, seed, tuber, root, flower |
| Musa acuminata | Leaf, pulp, fruit, bulb, seed, tuber, root, flower |
| Nepenthes khasiana | Leaf, pulp, fruit, bulb, seed, tuber, root, flower |
| Oryza rufipogon | Leaf, pulp, fruit, bulb, seed, tuber, root, flower |
| Oryza sativa | Leaf, pulp, fruit, bulb, seed, tuber, root, flower |
| Panicum virgatum cv. Trailblazer | Leaf, pulp, fruit, bulb, seed, tuber, root, flower |
| Paris polyphylla var. yunnanensis | Leaf, pulp, fruit, bulb, seed, tuber, root, flower |
| Picea sitchensis | Leaf, pulp, fruit, bulb, seed, tuber, root, flower |
| Pogostemon cablin | Leaf, pulp, fruit, bulb, seed, tuber, root, flower |
| Polygonatum odoratum | Leaf, pulp, fruit, bulb, seed, tuber, root, flower |
| Populus trichocarpa | Leaf, pulp, fruit, bulb, seed, tuber, root, flower |
| Psidium guajava | Leaf, pulp, fruit, bulb, seed, tuber, root, flower |
| Quercus suber | Leaf, pulp, fruit, bulb, seed, tuber, root, flower |
| Saussurea costus | Leaf, pulp, fruit, bulb, seed, tuber, root, flower |
| Senna alexandrina | Leaf, pulp, fruit, bulb, seed, tuber, root, flower |
| Selaginella moellendorffii | Leaf, pulp, fruit, bulb, seed, tuber, root, flower |
| Setaria italica | Leaf, pulp, fruit, bulb, seed, tuber, root, flower |
| Silene conica | Leaf, pulp, fruit, bulb, seed, tuber, root, flower |
| Solanum tuberosum | Leaf, pulp, fruit, bulb, seed, tuber, root, flower |
| Solidago canadensis | Leaf, pulp, fruit, bulb, seed, tuber, root, flower |
| Tanacetum parthenium | Leaf, pulp, fruit, bulb, seed, tuber, root, flower |
| Taxus x media | Leaf, pulp, fruit, bulb, seed, tuber, root, flower |
| Triticum aestivum | Leaf, pulp, fruit, bulb, seed, tuber, root, flower |
| Valeriana officinalis | Leaf, pulp, fruit, bulb, seed, tuber, root, flower |
| Viburnum davidii | Leaf, pulp, fruit, bulb, seed, tuber, root, flower |
| Vitis vinifera | Leaf, pulp, fruit, bulb, seed, tuber, root, flower |
| Zea mays | Leaf, pulp, fruit, bulb, seed, tuber, root, flower |
| Zingiber officinale | Leaf, pulp, fruit, bulb, seed, tuber, root, flower |

The amounts of costunolide, or other compounds of interest such as those described herein, present in certain extracts (% of extract, w/w) will be determined. Quantification of the compounds will be performed by normalizing the results by the weight of the extracts. The results of these analyses will be determined.

### Example 3

An oral composition is provided on a daily basis, prior to going to sleep with the intent on a full night's rest, the oral composition utilizing a caplet formulation comprising an extract of Eupatorium perfoliatum and a carrier.

Directions: As an oral composition, 1 capsule is orally administered with an 8 oz. glass of water once daily prior to going to sleep. Preferably, the oral composition is consumed with the intent of a full night's sleep.

### Example 4

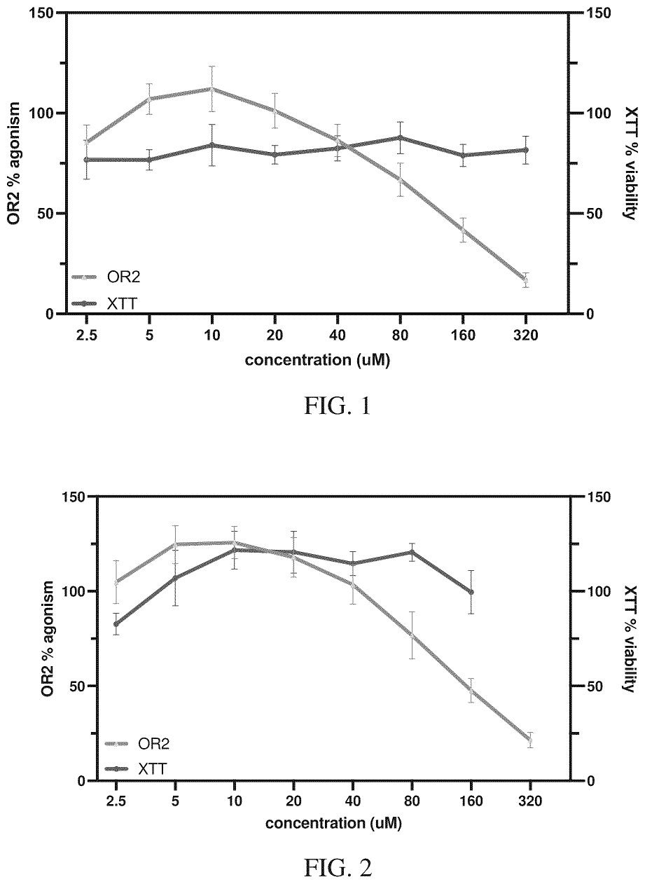

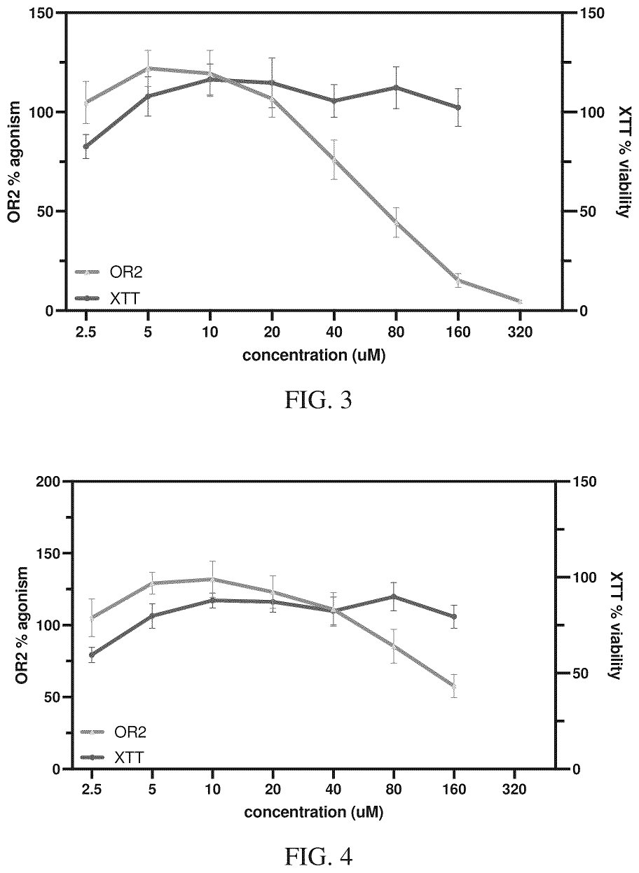

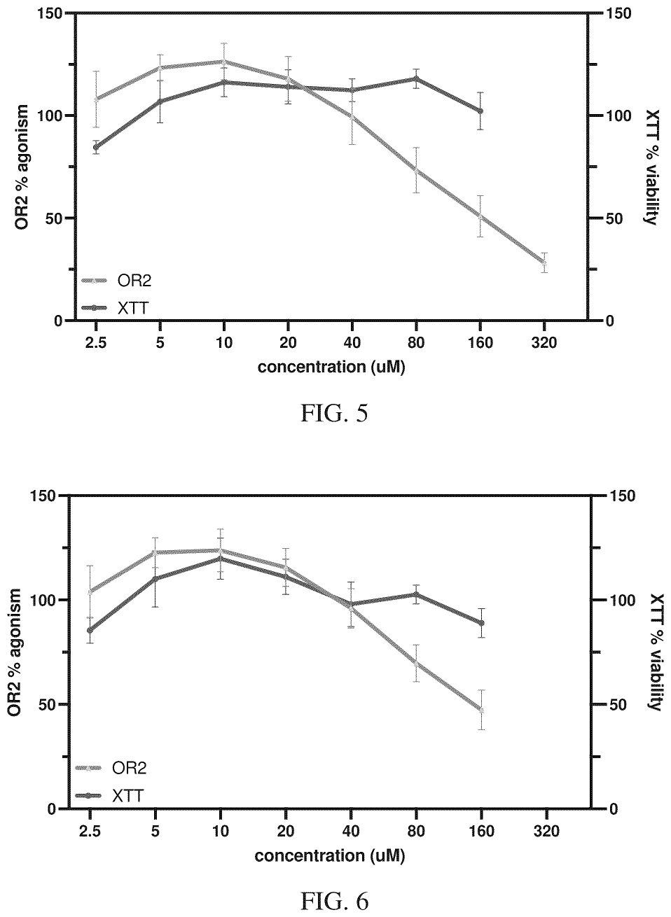

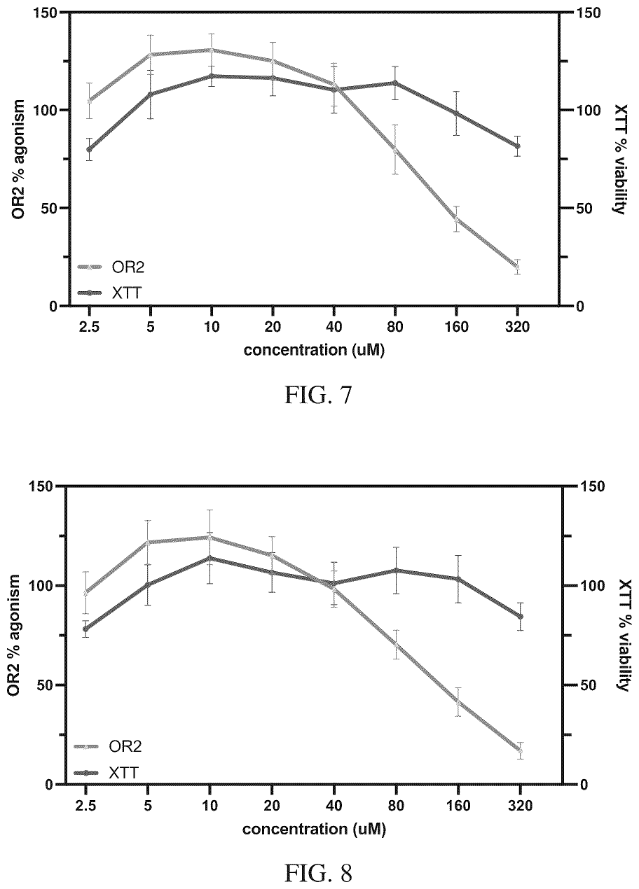

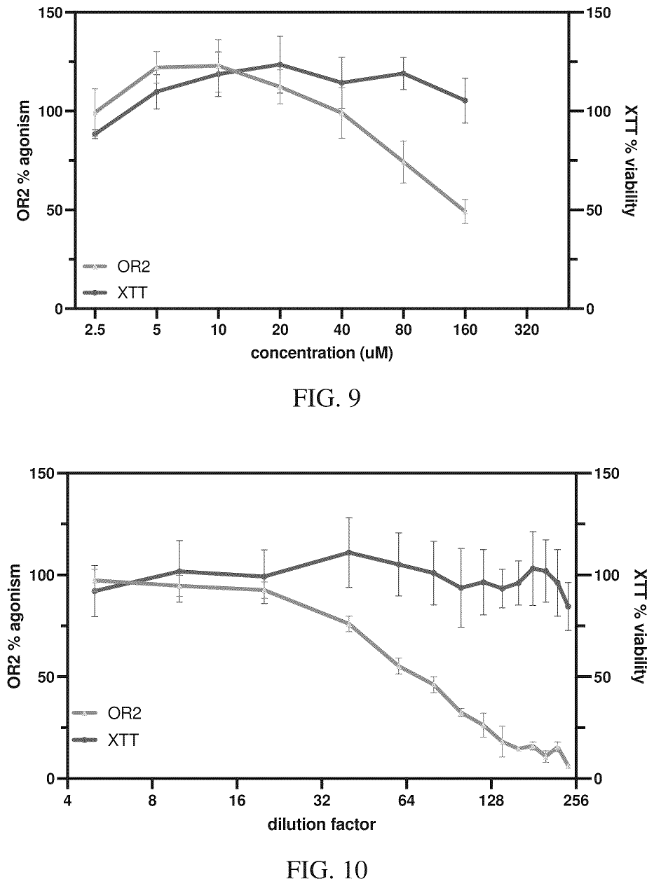

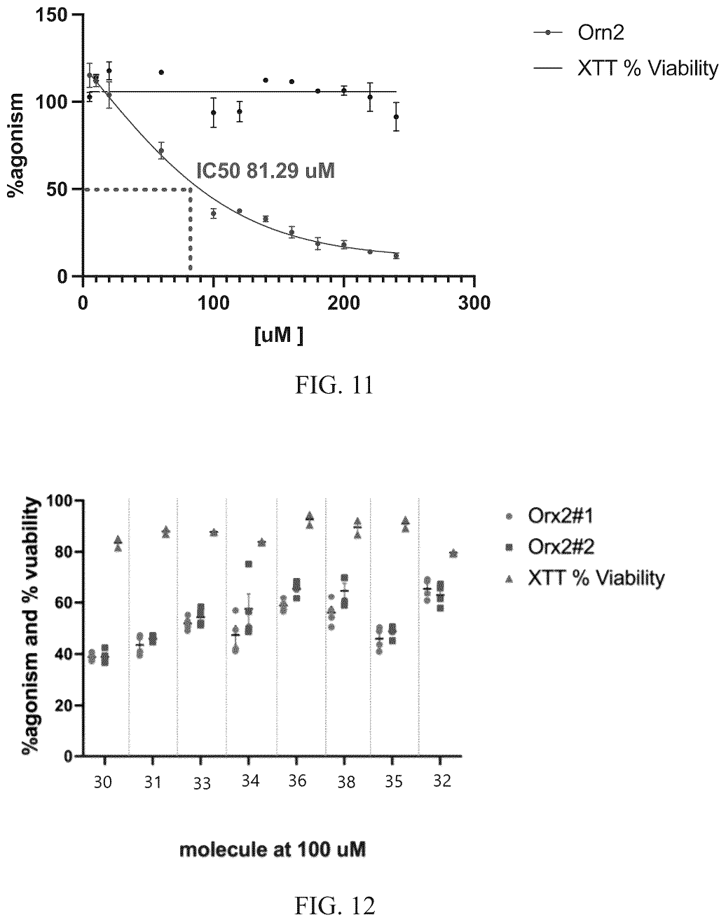

In this example, in vitro bioactivity of naturally occurring compounds from the plants described herein were tested to determine if they were capable of modulating an Orexin receptor. Structures 20-38 were identified as modulating an Orexin receptor (Orx1, Orx2) or both. FIGS. 1-11 illustrate compounds 20-30 modulating Orx2 agonism. FIG. 12 illustrates compounds 31-38 modulating Orexin receptor (Orx1, Orx2) agonism.

### Example 5

In this example, in vitro bioactivity of naturally occurring compounds was assessed.

For this example, the following protocol was used: 1.) seed cells in 20 μL Cell Plating Reagent; 2.) incubate cells overnight at 37° C. and 5% CO₂; 3.) treat cells with 2.5 μL 10× antagonist prepared in Compound Dilution Buffer (For OR2 screening, 2.5 μL of 1:100 natural product sample in DiscoverX cell plating reagent. Control antagonist, Seltorexant, 10× stock was prepared at 1500 nM); 4.) incubate for 30 minutes at 37° C. and 5% CO₂; 5.) treat cells with 2.5 μL 10× agonist prepared in Compound Dilution Buffer (For OR2 screening, 2.5 μL of 10× Orexin B EC80, 252.7 nM); 6.) incubate for 90 minutes at 37° C. and 5% CO₂; 7.) add 12.5 μL Working Detection Solution; 8.) incubate for 1 hour at room temperature in the dark; and 9.) read chemiluminescent signal.

The plants described herein were tested to determine the concentration, in μM, to achieve the relative IC50 of OR2 receptor, the absolute percent agonism achieved at the μM concentration of the relative OR2 IC50, the percentage receptor activity at the concentration, in μM, needed to achieve IC50 in OR2, the percentage cell viability, measured by XTT assay, at the IC50 μM concentration, the concentration of the fraction, in fold dilution, needed to achieve IC50 of OR2, and the percentage receptor activity at the concentration of the fraction, in fold dilution, needed to achieve IC50 of OR2. The results are described in Table 2.

TABLE 2

| | | | % | % | | | |
|---|---|---|---|---|---|---|---|
| | | Relative | Agonism | Agonism | % | IC50 | % |
| | | IC50 of | Orx2 at | on Orx1 | Cell | Dilution of | Cell |
| Cmpd | Plant | Orx2 | Relative | at OR2 | viability | Orx2 (fold | viability |
| ID | Source | (μM) | IC50 | IC50 | at IC50 | dilution) | at IC50 |
| 12 | Arnica | 53.34 | 65 | 64.4 | 80.39 | 7039.1 | 61.32 |
| | Montana | | | | | | |
| 13 | Eupatorium | 72.46 | 50 | 42.72 | 98.24 | 2283.9 | 88.38 |
| | perfolatum | | | | | | |
| 14 | Tanatecum | 88.99 | 50 | 62.98 | 94.58 | 3466.5 | 76.05 |
| | parthenium | | | | | | |
| 15 | Eupatorium | 79.1 | 65 | 67.71 | 103.46 | 2283.9 | 88.38 |
| | perfoliatum | | | | | | |
| 16 | Viburnum | 381.16 | 75 | 77.95 | 85.11 | 5786.9 | 93.99 |
| | davidii | | | | | | |
| 17 | Viburnum | 188.01 | 80 | 100.52 | 89.87 | 5786.9 | 93.99 |
| | davidii | | | | | | |
| 19 | Viburnum | 318.56 | 80 | 68.74 | 86.12 | 5786.9 | 93.99 |
| | davidii | | | | | | |
| 20 | Clausena | 146.07 | 45 | N/A | 83.58 | N/A | N/A |
| | excavata | | | | | | |
| 21 | Gynura | 177.51 | 45 | 42.54193 | 94.95 | N/A | N/A |
| | procumbens | | | | | | |
| 22 | Eruca | 84.7 | 40 | 63.03882 | 101.13 | N/A | N/A |
| | vesicaria | | | | | | |
| 23 | Senna | 174.06 | 50 | 104.1268 | 107.68 | N/A | N/A |
| | alexandrina | | | | | | |
| 24 | Clausena | 191.35 | 45 | 48.82759 | 96.12 | N/A | N/A |
| | excavata | | | | | | |
| 25 | — | 147.76 | 50 | 103.7053 | 91.68 | N/A | N/A |
| 26 | Senna | 178.73 | 40 | 60.57486 | 98.95 | N/A | N/A |
| | alexandrina | | | | | | |
| 27 | Psidium | 153.63 | 45 | 56.17954 | 101.69 | N/A | N/A |
| | guajava | | | | | | |
| 28 | — | 153.58 | 50 | 75.73956 | 107.58 | N/A | N/A |
| 30 | Tanacetum | 81.29 | 50 | 26.23297 | 100 | | |
| | parthenium | | | | | | |

### Example 6

In this example, the in vivo tolerability of Compounds 26, 21, 29, and 13 was assessed.

Naïve rats (male Sprague-Dawley rat) were acclimatized to the procedure room in their home cages up to 7 days following arrival to the test facility, with food and water available ad libitum. On the day of experiment, animals were acclimatized to the experimental room for at least one hour prior to the beginning of any experiment. All animals were tail marked, weighed and randomly assigned to treatment groups. The animals (n=3 per dose) were treated with either Compounds 26, 21, 29, and 13 (10 mL/kg, p.o.). For each compound a maximum of 4 doses were tested using a dose escalation procedure starting from 30 mg/kg. If this initial dose is well-tolerated, the dosage is increased to 100 mg/kg. If the 30 mg/kg dose is not tolerated, the dose is reduced to 15 mg/kg. Following the dose escalation procedure, if the 100 mg/kg dose is well-tolerated, the dose is escalated to 315 mg/kg. If it is not tolerated, the dose is decreased to 60 mg/kg. The pattern continues with the next decision point: if 315 mg/kg is well-tolerated, the dose is increased to the maximum of 500 mg/kg. If 315 mg/kg is not tolerated, the dose is decreased to 200 mg/kg. Following the administration of the compounds, each animal was placed in a clear Perspex cage, and was observed for 60 min of which the recordings were taken at 15 min, 30 min and 60 min time points.

Compound 26 was supplied from Arctom as a yellow powder. For formulation, a vehicle of 10% Dimethyl Sulfoxide (DMSO) in 20% Hydroxypropyl Beta Cyclodextrin (HPBC) was used. The dosing escalation procedure involved a dose volume of 10 mL/kg, based on an assumed body weight of 300 grams per animal. This results in a dose volume of approximately 3 mL per animal. With 3 animals per group, the total dose volume required per group is 9 mL. Four dose concentrations were prepared: For the 30 mg/kg dose concentration, 45 mg of Compound 26 was weighed and dissolved in a total volume of 15 mL (1.5 mL DMSO+13.5 mL 20% HPBC). After weighing, the Compound 26 was dissolved in the calculated volume of the vehicle. To ensure proper mixing and dissolution, the solution was sonicated, resulting in a yellow milky solution. A similar procedure was followed for the other doses.

Compound 21 was supplied from Brightseed, Inc as a yellow powder. For formulation, a vehicle of 10% Dimethyl Sulfoxide (DMSO) in 20% Hydroxypropyl Beta Cyclodextrin (HPBC) was used. Four dose concentrations were prepared: For the 30 mg/kg dose concentration, 45 mg of Tiliroside was weighed and dissolved in a total volume of 15 mL (1.5 mL DMSO+13.5 mL 20% HPBC). To ensure proper mixing and dissolution, the solution was sonicated, resulting in a yellow milky solution. A similar procedure was followed for the other doses.

Compound 29 was supplied from Brightseed, Inc. as an orange powder. For formulation, a vehicle of 10% Dimethyl Sulfoxide (DMSO) in 20% Hydroxypropyl Beta Cyclodextrin (HPBC) was used. For the 30 mg/kg dose concentration, 45 mg of Compound 29 was weighed and dissolved in a total volume of 15 mL (1.5 mL DMSO+13.5 mL 20% HPBC). After weighing, the Compound 29 was dissolved in the calculated volume of the vehicle. To ensure proper mixing and dissolution, the solution was sonicated and 10 μL of NaOH was added at a time and then sonicated again until no solid matter remained, resulting in a thick dark red solution. A similar procedure was followed for all doses.

Compound 13 was supplied as a yellow powder from Ambeed, Inc. For formulation, a vehicle of 10% Dimethyl Sulfoxide (DMSO) in 20% Hydroxypropyl Beta Cyclodextrin (HPBC) was used. For the 30 mg/kg dose concentration, 45 mg of Compound 13 was weighed and dissolved in a total volume of 15 mL (1.5 mL DMSO+13.5 mL 20% HPBC). After weighing, the Compound 13 was dissolved in the calculated volume of the vehicle. To ensure proper mixing and dissolution, the solution was sonicated and 10 μL of NaOH was added at a time and then sonicated again until no solid matter remained, resulting in a bright yellow solution. A similar procedure was followed for all doses.

The compounds would be considered not tolerated if three or more adverse effects (or any severe adverse effect) occurred at specific dose during the entire period of 60 mins. Animals were observed for adverse effects such as marked piloerection, abnormal breathing, hunched, vocalization, reduced activity etc. following administration of the compound. Reduced activity is determined when the animal shows decreased activity and stops moving in the cage without sleeping. Mild—slight decrease in activity, responsive when provoked. Moderate—decrease in activity, less responsive when provoked. Pica indicates that the animal was eating non-food items such as beddings. The results of Example 5 are summarized in Table 3 below.

TABLE 3
Adverse effect profiles of different compounds at different time points.

| | Dose | | | |
|---|---|---|---|---|
| Treatment | (mg/kg) | 15 min effects | 30 min effects | 60 min effects |
| Compound | 30 | Reduced activity | No adverse effects | Reduced activity (3/3) |
| 26 | | (3/3) | | |
| | 100 | Reduced activity | Reduced activity | Reduced activity (3/3) |
| | | (1/3) | (2/3) | |
| | 315 | No adverse effects | No adverse effects | No adverse effects |
| | 500 | No adverse effects | Reduced activity | No adverse effects |
| | | | (3/3) | |
| Compound | 30 | Reduced activity | No adverse effects | Reduced activity (3/3) |
| 21 | | (2/3) | | |
| | 100 | No adverse effects | Reduced activity | Reduced activity (3/3) |
| | | | (1/3) | |
| | 315 | No adverse effects | No adverse effects | No adverse effects |
| | 500 | No adverse effects | Reduced activity | Pica (2/3) |
| | | | (2/3) | |
| Compound | 30 | No adverse effects | No adverse effects | No adverse effects |
| 29 | 100 | Reduced activity | No adverse effects | No adverse effects |
| | | (3/3) | | |
| | 315 | Reduced activity | Reduced activity | Reduced activity (3/3) |
| | | (2/3) | (2/3) | |
| | | | Pica (2/3) | |
| | 500 | Pica (1/3) | Pica (2/3) | No adverse effects |
| Compound | 30 | No adverse effects | No adverse effects | Reduced activity (3/3) |
| 13 | 100 | No adverse effects | Reduced activity | No adverse effects |
| | | | (1/3) | |
| | 315 | No adverse effects | No adverse effects | No adverse effects |
| | 500 | Pica (2/3) | Reduced activity | Reduced activity (1/3) |
| | | | (2/3) | |
| | | | Pica (2/3) | |

In summary, all animals dosed with Compounds 26, 21, 29, and 13 at all four doses (30, 100, 315 and 500 mg/kg) can be considered well-tolerated. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C“would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. [0181] While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An oral composition for improving, restoring, modulating, or maintaining sleep, the oral composition comprising:
a compound of Formula (I); 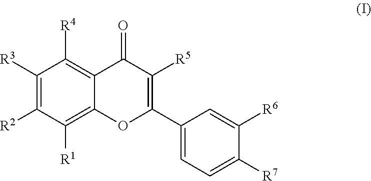
wherein R¹ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R² is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R³ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R⁴ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R⁵ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R⁶ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R⁷ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
L is —Z¹-Z² or —Z¹-Z²—Z³;
Z₁ is —CH₂—, —O—, —NH—, optionally substituted C₃-C₈ cycloalkyl, optionally substituted C₆ to C₁₀ aryl, optionally substituted —C₃-C₈ heterocyclyl, or optionally substituted C₃ to C₁₀ heteroaryl. In some embodiments, Z1 is optionally substituted —C₃-C₈ heterocyclyl;
Z² is hydrogen, deuterium, halo, —CH₂—, —O—, —CO₂H, —CO₂CHCH—, optionally substituted —C₃-C₈ cycloalkyl, optionally substituted —C₆-C₁₀ aryl, optionally substituted —C₃-C₈ heterocyclyl, or optionally substituted —C₃-C₁₀ heteroaryl;
Z³ is hydrogen, deuterium, halo, —CH₂—, —O—, optionally substituted —C₃-C₈ cycloalkyl, optionally substituted —C₆-C₁₀ aryl, optionally substituted —C₃-C₈ heterocyclyl, or optionally substituted —C₃-C₁₀ heteroaryl; and
one or more carriers or excipients.

2. The oral composition of claim 1, wherein the compound is represented by Formula (IA), or a salt thereof:

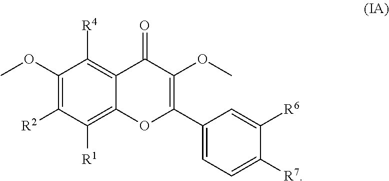

3. The oral composition of claim 1, wherein the compound is represented by Formula (IB), or a salt thereof:

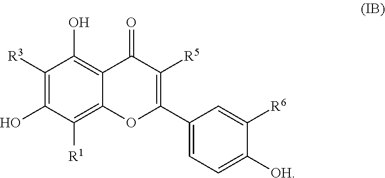

4. The oral composition of any one of claims 1 to 3, wherein the compound is derived from a plant extract.

5. The oral composition of claim 4, wherein the plant extract is selected from the group consisting of Eupatorium perfoliatum, Zingiber officinale, Picea sitchensis, Viburnum davidii, Arnica montana, Helianthus annuus, Lactuca sativa, Barnadesia spinosa, Dolomiaea souliei, Saussurea costus, Artemisia annua, Inula helenium, Laurus nobilis, Momodica cochinchinensis, Pogostemon cablin, Tanacetum parthenium, Cichorium intybus, Solidago Canadensis, Horeum vulgare, Tricum aestivum, Silene conica, Alloteropsis semialata, Paris polyphylla, Camella sinensis, Glebionis cornaria, Hordeum vulgare, Setaria italic, Zea mays, Arabidopsis thaliana, Brachypodium distachyon, Grindelia integrifolia, Lobelia chinesis, Arachis hypogaea, Nepenthes khasiana, Selaginella mollendorffii, Taxus×media, Acorus gramieus, Andrographis paniculata, Artemisia maritima, Bletilla striata, Fallopia mulfora, Gossypium hirsutum, Hordeum spontaneum, Hypericum perforatum, Musa acuminate, Oryza rufipogon, Oryza sativa, Quercus suber, Solanum tuberosum, Valeriana officinallis, Vitis vinifera, Polygonatum odoratum, Glycine max, Panicum virgatum cv. Trailblazer, Clausena excavata, Gynura procumbens, Psidium guajava, Eruca vesicaria, Senna alexandrina, or Populus trichorarpa.

6. The oral composition of claim 4 or 5, wherein the plant extract further comprises a compound selected from the group consisting of costunolide, 5a,9-dimethyl-3-methylidene-3ah,4h,5h,9bh-naphtho[1,2-b]furan-2,8-dione, Baynol A, (3r,3ar,4r,11as)-3,6,10-trimethyl-2-oxo-3h,3ah,4h,7h,8h,11h,11ah-cyclodeca[b]furan-4-yl acetate, (3r,3as,4r,11as)-4-hydroxy-3,6,10-trimethyl-3h,3ah,4h,7h,8h,11h,11ah-cyclodeca[b]furan-2-one, 1R,2S,5S,6S,7S)-5-isopropyl-2,8-dimethyltricyclo[4.4.0.0^{2,7}]dec-8-en-4-one, 2-((1R,3S,4R)-4-methyl-3-(prop-1en-2-yl)-4-vinylcyclohexyl)acrylaldehyde, 3,7,11-trimethyldodeca-1,3,5,8,10-pentaene, p-cymene, (1S,2R,4R,7E,11S)-4,8-dimethyl-12-methylidene-3,14-dioxatricyclo[9.3.0.02,4]tetradec-7-en-13-one, 3-[(3aS,4R,5R,8aR)-4-hydroxy-5,7-dimethyl-3-methylidene-2-oxo-4,5,8,8a-tetrahydro-3aH-cyclohepta[b]furan-6-yl]propyl acetate, 3,5,7-trihydroxy-2-(4-hydroxyphenyl)chromen-4-one, 2-(3,4-dihydroxyphenyl)-5,7-dihydroxychromen-4-one, 5,7-dihydroxy-2-(4-hydroxyphenyl)-3,6-dimethoxychromen-4-one, 2-(3,4-dihydroxyphenyl)-5,7-dihydroxy-3-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one, 1-(2-hydroxy-4-methoxyphenyl)-3-(4-hydroxyphenyl)propan-1-one, 3-(4-hydroxyphenyl)-1-[4-hydroxy-2-[3,4,5-trihydroxy-6-(hydroxymethyl)oxan-2-yl]oxyphenyl]propan-1-one, 3-(4-hydroxyphenyl)-1-[4-methoxy-2-[3,4,5-trihydroxy-6-(hydroxymethyl)oxan-2-yl]oxyphenyl]propan-1-one, 3-(4-hydroxyphenyl)-1-[2-hydroxy-4-[3,4,5-trihydroxy-6-(hydroxymethyl)oxan-2-yl]oxyphenyl]propan-1-one, (2S,3S,4S,5R,6S)-6-[5,7-dihydroxy-2-(4-hydroxyphenyl)-4-oxochromen-3-yl]oxy-3,4,5-trihydroxyoxane-2-carboxylic acid, 7-[(2S,3R,4S,5S,6R)-4,5-dihydroxy-6-(hydroxymethyl)-3-[(2S,3R,4R,5R,6S)-3,4,5-trihydroxy-6-methyloxan-2-yl]oxyoxan-2-yl]oxy-3,5-dihydroxy-2-(4-hydroxyphenyl)chromen-4-one, 3-[(2S,3R,4S,5S,6R)-4,5-dihydroxy-6-(hydroxymethyl)-3-[(2S,3R,4R,5R,6S)-3,4,5-trihydroxy-6-methyloxan-2-yl]oxyoxan-2-yl]oxy-5,7-dihydroxy-2-(4-hydroxyphenyl)chromen-4-one, 3-[(2S,5R)-3,5-dihydroxy-6-(hydroxymethyl)-4-[(2S,3S,5R)-3,4,5-trihydroxy-6-methyloxan-2-yl]oxyoxan-2-yl]oxy-5,7-dihydroxy-2-(4-hydroxyphenyl)chromen-4-one, [(2R,3S,4S,5R,6S)-6-[3,5-dihydroxy-2-(4-hydroxy-3-methoxyphenyl)-8-methoxy-4-oxochromen-7-yl]oxy-3,4-dihydroxy-5-[(2S,3R,4R,5R,6S)-3,4,5-trihydroxy-6-methyloxan-2-yl]oxyoxan-2-yl]methyl acetate, 5,7-dihydroxy-2-(4-hydroxyphenyl)-3-[(2S,3R,4S,5S)-3,4,5-trihydroxyoxan-2-yl]oxychromen-4-one, 3-[(2S,3R,4R,5S)-3,4-dihydroxy-5-(hydroxymethyl)oxolan-2-yl]oxy-5,7-dihydroxy-2-(4-hydroxyphenyl)chromen-4-one, (2S,3S,4S,5R,6S)-6-[4-(5,7-dihydroxy-3,6-dimethoxy-4-oxochromen-2-yl)-2-hydroxyphenoxy]-3,4,5-trihydroxyoxane-2-carboxylic acid, 2-(3,4-dihydroxyphenyl)-3,5,7-trihydroxychromen-4-one, 1,6-dihydroxy-3-methyl-8-[(2S,3R,4S,5S,6R)-3,4,5-trihydroxy-6-(hydroxymethyl)oxan-2-yl]oxyanthracene-9,10-dione, 1,3,8-trihydroxy-6-methylanthracene-9,10-dione, (E)-3-(4-hydroxyphenyl)-N-[4-[[(E)-3-(4-hydroxyphenyl)prop-2-enoyl]-[3-[[(E)-3-(4-hydroxyphenyl)prop-2-enoyl]amino]propyl]amino]butyl]prop-2-enamide, 5,7-dihydroxy-6-methoxy-2-(4-methoxyphenyl)chromen-4-one, 2-(3,4-dihydroxyphenyl)-5,7-dihydroxy-3-[(2S,3R,4S,5S,6R)-3,4,5-trihydroxy-6-(hydroxymethyl)oxan-2-yl]oxychromen-4-one, 2-(3,4-dihydroxyphenyl)-5,7-dihydroxy-6-methoxychromen-4-one, (E)-3-(4-hydroxyphenyl)-N-[3-[[(E)-3-(4-hydroxyphenyl)prop-2-enoyl]-[4-[[(E)-3-(4-hydroxyphenyl)prop-2-enoyl]-[3-[[(E)-3-(4-hydroxyphenyl)prop-2-enoyl]amino]propyl]amino]butyl]amino]propyl]prop-2-enamide, (1S,3R,4R,5R)-3,4-bis[[(E)-3-(3,4-dihydroxyphenyl)prop-2-enoyl]oxy]-1,5-dihydroxycyclohexane-1-carboxylic acid, and 5,7-dihydroxy-2-(4-hydroxyphenyl)chromen-4-one.

7. The oral composition of any one of claims 1 to 6, wherein the oral composition is formulated as a dietary supplement, food ingredient or additive, a medical food, nutraceutical or pharmaceutical composition.

8. The oral composition of any one of claims 1 to 7, wherein the oral composition is in a dosage form configured as one of a soft-gel capsule, a hard capsule, liquid, powder, and a tablet.

9. The oral composition of any one of claims 1 to 8, wherein the oral composition comprises 0.01% to 99% w/w of the compound of Formula (I).

10. The oral composition of any one of claims 4 to 9, wherein the oral composition comprises 0.01% to 99% w/w of the plant extract.

11. The oral composition of any one of claims 1 to 10, wherein the oral composition further comprises a preservative.

12. The oral composition of any one of claims 1 to 11, wherein the oral composition further comprises a sweetener.

13. The oral composition of any one of claims 1 to 12, wherein the oral composition further comprises a coloring agent.

14. The oral composition of any one of claims 1 to 13, wherein the oral composition further comprises an additive.

15. The oral composition of any one of claims 1 to 14, wherein the oral composition further comprises a flavoring agent.

16. The oral composition of any one of claims 1 to 15, wherein the compound of Formula (I) consists essentially of Compounds 12, 13, 14, 15, 20, 21, 22, 23, 24, 25, 26, 27, 28, 29, 33, 34, 35, and 38 and is unbound to a lignocellulosic material.

17. The oral composition of any one of claims 1 to 16, with a proviso that the oral composition does not comprise lignin, cellulose, or pectin.

18. The oral composition of any one of claims 1 to 3, with a proviso that the compound of Formula (I) is not an extract from a natural source.

19. The oral composition of any one of claims 1 to 18, wherein the compound of Formula (I) is selected from Compounds 14, 22, 23, and 26.

20. The oral composition of claim 19, wherein the compound of Formula (I) is selected from Compounds 22 and 23.

21. The oral composition of any one of claims 1 to 20, wherein the oral composition is formulated as a solid.

22. The oral composition of claim 21, wherein the solid is a food product, tablet or capsule.

23. The oral composition of claim 11, wherein the preservative is from about 0.01% to about 5% by weight of the composition.

24. The oral composition of any one of claims 1 to 23, wherein the oral composition further comprises a compound of Formula (II): 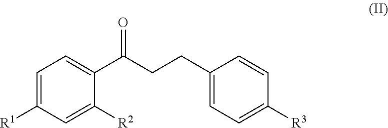
or a salt thereof, wherein,
R¹ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R² is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R³ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
L is —Z¹—Z² or —Z¹-Z²—Z³;
Z₁ is —CH₂—, —O—, —NH—, optionally substituted C₃-C₈ cycloalkyl, optionally substituted C₆ to C₁₀ aryl, optionally substituted —C₃-C₈ heterocyclyl, or optionally substituted C₃ to C₁₀ heteroaryl. In some embodiments, Z1 is optionally substituted —C₃-C₈ heterocyclyl;
Z² is hydrogen, deuterium, halo, —CH₂—, —O—, —CO₂H, —CO₂CHCH—, optionally substituted —C₃-C₈ cycloalkyl, optionally substituted —C₆-C₁₀ aryl, optionally substituted —C₃-C₈ heterocyclyl, or optionally substituted —C₃—C toheteroaryl; and
Z³ is hydrogen, deuterium, halo, —CH₂—, —O—, optionally substituted —(C₃-C₈ cycloalkyl, optionally substituted —C₆-C₁₀ aryl, optionally substituted —C₃-C₈ heterocyclyl, or optionally substituted —C₃-C₁₀ heteroaryl.

25. The oral composition of claim 24, wherein the compound of Formula (II) is Compound 16, 17, 18, or 19.

26. The oral composition of any one of claims 1 to 25, wherein the oral composition further comprises a compound of Formula (III) or a salt thereof: 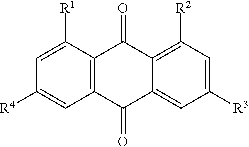
wherein R¹ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R² is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R³ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
R⁴ is selected from H, deuterium, hydroxyl, halogen, cyano, optionally substituted amino, optionally substituted —C₁-C₆ alkyl, optionally substituted —O—C₁-C₆ alkyl, optionally substituted —C₃-C₈ cycloalkyl, optionally substituted —O—C₃-C₈ cycloalkyl, optionally substituted —C₃-C₈ heteroalkyl, optionally substituted —O—C₃-C₈ heteroalkyl, optionally substituted —C₆-C₁₀ aryl, optionally substituted —O—C₆-C₁₀ aryl, or L;
L is —Z¹—Z² or —Z¹-Z²—Z³;
Z₁ is —CH₂—, —O—, —NH—, optionally substituted C₃-C₈ cycloalkyl, optionally substituted C₆ to C₁₀ aryl, optionally substituted —C₃-C₈ heterocyclyl, or optionally substituted C₃ to C₁₀ heteroaryl. In some embodiments, Z1 is optionally substituted —C₃-C₈ heterocyclyl;
Z² is hydrogen, deuterium, halo, —CH₂—, —O—, —CO₂H, —CO₂CHCH—, optionally substituted —C₃-C₈ cycloalkyl, optionally substituted —C₆-C₁₀ aryl, optionally substituted —C₃-C₈ heterocyclyl, or optionally substituted —C₃-C₁₀ heteroaryl; and
Z³ is hydrogen, deuterium, halo, —CH₂—, —O—, optionally substituted —(C₃-C₈ cycloalkyl, optionally substituted —C₆-C₁₀ aryl, optionally substituted —C₃-C₈ heterocyclyl, or optionally substituted —C₃-C₁₀ heteroaryl.

27. The oral composition of claim 26, wherein the compound of Formula (III) is Compound 30 or 31.

28. The oral composition of any one of claims 1 to 27, wherein the oral composition comprises an orexin receptor 2 antagonist.

29. The oral composition of any one of claims 1 to 28, wherein the oral composition an orexin receptor 1 antagonist.

30. A method improving, restoring, modulating, or maintaining sleep in a subject in need thereof, the method comprising:
administering an oral composition of any one of claims 1 to 29,
wherein the oral composition is administered in an amount sufficient to improve, restore, modulate, or maintain sleep in the subject in need thereof.

31. The method of claim 30, wherein administering the oral composition further provides a decrease in pERK1/2 phosphorylation.

32. The method of claim 30 or 31, wherein administering the oral composition further provides a decrease in calcium influx.

33. The method of any one of claims 30 to 32, wherein improving sleep provides a subject a full night's sleep.

34. The method of any one of claims 30 to 33, wherein improving sleep provides a subject a restful night's sleep.

35. The method of any one of claims 30 to 34, wherein modulating sleep provides a subject an increase in Slow-Wave-Sleep 1-4.

36. The method of any one of claims 30 to 35, wherein modulating sleep provides a subject an increase in REM sleep.

37. The method of any one of claims 30 to 36, wherein modulating sleep decreases the time to sleep onset in the subject.

38. The method of claim 37, wherein the time to sleep onset is from about 15 minutes to about 180 minutes.

39. The method of any one of claims 30 to 38, wherein modulating sleep increases the average sleep length in the subject.

40. The method of claim 39, wherein the average sleep length is increased from about 15 minutes to about 180 minutes.

41. The method of any one of claims 30 to 40, wherein the oral composition is administered to the subject prior to sleep.

42. A method of treating a sleeping disorder in a subject in need thereof, the method comprising:
administering a therapeutically effect amount of an oral composition of any one of claims 1 to 29,
thereby treating a sleeping disorder in a subject in need thereof.

43. The method of claim 42, wherein administering the oral composition further provides a decrease in pERK1/2 phosphorylation.

44. The method of claim 42 or 43, wherein administering the oral composition further provides a decrease in calcium influx.

45. The method of any one of claims 42 to 44, wherein the sleep disorder is selected from the group consisting of insomnias, sleep related breathing disorders, central disorders of hypersomnolence, circadian rhythm sleep-wake disorders, parasomnias, and sleep related movement disorders.

46. The method of any one of claims 42 to 45, wherein the sleep disorder is selected from the group consisting of insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, and sleeping sickness.

47. The method of any one of claims 42 to 46, wherein an orexin receptor is activated or restored after administration of the oral composition.

48. The method of any one of claims 42 to 47, wherein the oral composition is administered to the subject prior to sleep.

49. The method of any one of claims 42 to 48, further comprising administering an additional compound to treat the sleep disorder.

50. The method of claim 49, wherein the additional compound to treat the sleep disorder is selected from the group consisting of sedatives, hypnotics, anxiolytics, antipsychotics, antianxiety agents, antihistamines, benzodiazepines, barbiturates, cyclopyrrolones, GABA agonists, 5HT-2 antagonists. histamine antagonists, imidazopyridines, minor tranquilizers, melatonin agonists and antagonists, melatonergic agents, prokineticin agonists and antagonists, pyrazolopyrimidines, T-type calcium channel antagonists, triazolopyridines, melatonin, mephobarbital, meprobamate, methaqualone, methyprylon, midaflur, midazolam, modafinil, nefazodone, NGD-2-73, nisobamate, nitrazepam, nortriptyline, nortriptyline, oxazepam, paraldehyde, paroxetine, pentobarbital, perlapine, perphenazine, phenelzine, phenobarbital, prazepam, promethazine, propofol, protriptyline, quazepam, ramelteon, reclazepam, roletamide, secobarbital, sertraline, suproclone, TAK-375, temazepam, thioridazine, tiagabine, tracazolate, tranylcypromaine, trazodone, triazolam, trepipam, tricetamide, triclofos, trifluoperazine, trimetozine, trimipramine, uldazepam, venlafaxine, zaleplon, zolazepam, zopiclone, zolpidem, and combinations thereof.
